SEQUENCE LISTING (64 sequences)

SEQ ID NO: 1 - protein, 295 aa, synthetic construct
SINPWILTGFADAEGSFMLRIRKRNKMSVGYLTELVFGITLHNKDKSILENIQSTWGVGVIYNSGDNAVSLRVTRFEDLKVIIDHFEKYPLITQKYADYMLFKQAFNVMENKEHLTIEGIKELVRIKAKLNWGLTDELKKAFPEIISKERSLINKNIPNFKWLAGFTSGEGCFFVALRKAKSKRGVQVYLRFSIGQHIRDKNLMNSLITYLGCGYIEKHNKSEFSWLEFRVYKFSDIRDKIIPFFQEYTLIGTKLKDFEDWCKVAKLIEEKKHLTEEGLDEIKKIKLNMNKGRVF

SEQ ID NO: 2 - protein, 295 aa, synthetic construct
SINPWILTGFADAEGSFMLRIRKRNKMSVGYLTELVFGITLHNKDKSILENIQSTWGVGVIYNSGDNAVSLRVTRFEDLKVIIDHFEKYPLITQKYADYMLFKQAFNVMENKEHLTIEGIKELVRIKARLNWGLTDELKKAFPEIISKERSLINKNIPNFKWLAGFTSGEGCFFVALRKTKFKLGVQVYLRFSIGQHIRDKNLMNSLITYLGCGYIEKHNKSEFSWLEFRVYKFSDIRDKIIPFFQEYTLIGTKLKDFEDWCKVAKLIEEKKHLTEEGLDEIKKIKLNMNKGRVF

SEQ ID NO: 3 - protein, 295 aa, synthetic construct
SINPWILTGFADAEGSFMLRIRKRNKMSVGYLTELVFGITLHNKDKSILENIQSTWGVGVIYNSGDNAVSLRVTRFEDLKVIIDHFEKYPLITQKYADYMLFKQAFNVMENKEHLTIEGIKELVRIKAKLNWGLTDELKKAFPEIISKERSLINKNIPNFKWLAGFTSGEGCFFVALRKNKHKSGVQVYLRFSIGQHIRDKNLMNSLITYLGCGYIEKHNKSEFSWLEFRVYKFSDIRDKIIPFFQEYTLIGTKLKDFEDWCKVAKLIEEKKHLTEEGLDEIKKIKLNMNKGRVF

SEQ ID NO: 4 - protein, 295 aa, synthetic construct
SINPWILTGFADAEGSFMLRIRKRNKMSVGYLTELVFGITLHNKDKSILENIQSTWGVGVIYNSGDNAVSLRVTRFEDLKVIIDHFEKYPLITQKYADYMLFKQAFNVMENKEHLTIEGIKELVRIKAKLNWGLTDELKKAFPEIISKERSLINKNIPNFKWLAGFTSGEGCFFVALRKRKSKDGVQVYLRFSIGQHIRDKNLMNSLITYLGCGYIEKHNKSEFSWLEFRVYKFSDIRDKIIPFFQEYTLIGTKLKDFEDWCKVAKLIEEKKHLTEEGLDEIKKIKLNMNKGRVF

SEQ ID NO: 5 - protein, 295 aa, synthetic construct
SINPWILTGFADAEGSFMLRIRKRNKMSVGYLTELVFGITLHNKDKSILENIQSTWGVGVIYNSGDNAVSLRVTRFEDLKVIIDHFEKYPLITQKYADYMLFKQAFNVMENKEHLTIEGIKELVRIKAKLNWGLTDELKKAFPEIISKERSLINKNIPNFKWLAGFTSGEGCFFVALRKSKRKIGVQVYLRFSIGQHIRDKNLMNSLITYLGCGYIEKHNKSEFSWLEFRVYKFSDIRDKIIPFFQEYTLIGTKLKDFEDWCKVAKLIEEKKHLTEEGLDEIKKIKLNMNKGRVF

SEQ ID NO: 6 - protein, 295 aa, synthetic construct
SINPWILTGFADAEGSFMLRIRKRNKMSVGYLTELVFGITLHNKDKSILENIQSTWGVGVIYNSGDNAVSLRVTRFEDLKVIIDHFEKYPLITQKYADYMLFKQAFNVMENKEHLTIEGIKELVRIKAKLNWGLTDELKKAFPEIISKERSLINKNIPNFKWLAGFTSGEGCFFVALRKSKQKMGVQVYLRFSIGQHIRDKNLMNSLITYLGCGYIEKHNKSEFSWLEFRVYKFSDIRDKIIPFFQEYTLIGTKLKDFEDWCKVAKLIEEKKHLTEEGLDEIKKIKLNMNKGRVF

SEQ ID NO: 7 - protein, 295 aa, synthetic construct
SINPWILTGFADAEGCFLLRIFKDNKMSVGYGTRLVFQIWLHNKDKSILENIQSTWGVGVIANFGDNAVSLRVSRFEDLKVIIDHFEKYPLITQKYADYMLFKQAFNVMENKEHLTIEGIKELVRIKAKLNWGLTDELKKAFPEIISKERSLINKNIPNFKWLAGFTSGDGCFYVNLKKQKGKLGVGVQLVFDITQHIRDKNLMNSLITYLGCGYINKFNKSEFSILNFRVTKFSDIRDKIIPFFQEYTLIGTKLKDFEDWCKVAKLIEEKKHLTEEGLDEIKKIKLNMNKGRVF

SEQ ID NO: 8 - protein, 295 aa, synthetic construct
SINPWILTGFADAEGCFLLRIFKDNKMSVGYGTRLVFQIWLHNKDKSILENIQSTWGVGVIANFGDNAVSLRVSRFEDLKVIIDHFEKYPLITQKYADYMLFKQAFNVMENKEHLTIEGIKELVRIKAKLNWGLTDELKKAFPEIISKERSLINKNIPNFKWLAGFTSGDGCFYVNLKKDKVKGGVGVQLVFDITQHIRDKNLMNSLITYLGCGYINKSNKSEFSVLNFRVTKFSDIRDKIIPFFQEYTLIGTKLKDFEDWCKVAKLIEEKKHLTEEGLDEIKKIKLNMNKGRVF

SEQ ID NO: 9 - protein, 295 aa, synthetic construct
SINPWILTGFADAEGCFLLRIFKDNKMSVGYGTRLVFQIWLHNKDKSILENIQSTWGVGVIANFGDNAVSLRVSRFEDLKVIIDHFEKYPLITQKYADYMLFKQAFNVMENKEHLTIEGIKELVRIKAKLNWGLTDELKKAFPEIISKERSLINKNIPNFKWLAGFTSGDGCFYVNLKKQKSKLGLGVQLVFDITQHIRDKNLMNSLITYLGCGYINKFNKSEFSTLNFRVTKFSDIRDKIIPFFQEYTLIGTKLKDFEDWCKVAKLIEEKKHLTEEGLDEIKKIKLNMNKGRVF

SEQ ID NO: 10 - protein, 295 aa, synthetic construct
SINPWILTGFADAEGCFLLRIFKNNKMSVGYGTRLVFQIWLHNKDKSILENIQSTWGVGVIANFGDNAVSLRVSRFEDLKVIIDHFEKYPLITQKYADYMLFKQAFNVMENKEHLTIEGIKELVRIKAKLNWGLTDELKKAFPEIISKERSLINKNIPNFKWLAGFTSGDGCFYVNLKKQKSKWGVGVQLVFDITQHIRDKNLMNSLITYLGCGYINKSNKSEFSTLNFRVTKFSDIRDKIIPFFQEYTLIGTKLKDFEDWCKVAKLIEEKKHLTEEGLDEIKKIKLNMNKGRVF

SEQ ID NO: 11 - protein, 295 aa, synthetic construct
SINPWILTGFADAEGCFLLRIYKDNKMSVGYGTRLVFQIWLHNKDKSILENIQSTWGVGVIANFGDNAVSLRVSRFEDLKVIIDHFEKYPLITQKYADYMLFKQAFNVMENKEHLTIEGIKELVRIKAKLNWGLTDELKKAFPEIISKERSLINKNIPNFKWLAGFTSGDGCFYVNLIKQKTKLGLRVQLVFDITQHIRDKNLMNSLITYLGCGYINKSNKSEFSVLNFRVTKFSDIRDKIIPFFQEYTLIGTKLKDFEDWCKVAKLIEEKKHLTEEGLDEIKKIKLNMNKGRVF

SEQ ID NO: 12 - protein, 295 aa, synthetic construct
SINPWILTGFADAEGCFLLRIFKNNKMSVGYGTRLVFQIWLHNKDKSILENIQSTWGVGVIANFGDNAVSLRVSRFEDLKVIIDHFEKYPLITQKYADYMLFKQAFNVMENKEHLTIEGIKELVRIKAKLNWGLTDELKKAFPEIISKERSLINKNIPNFKWLAGFTSGDGCFYVNLKKEKGKWGLHVQLVFDITQHIRDKNLMNSLITYLGCGYINKSNKSEFSTLHFRVTKFSDIRDKIIPFFQEYTLIGTKLKDFEDWCKVAKLIEEKKHLTEEGLDEIKKIKLNMNKGRVF

SEQ ID NO: 13 - DNA, 22 bp, Herpes simplex virus-2
  source 1..22: mol_type = genomic DNA
tatcctttttttctaggtgttt

SEQ ID NO: 14 - DNA, 22 bp, Herpes simplex virus-2
  source 1..22: mol_type = genomic DNA
tctgcatctatacctctttctg

SEQ ID NO: 15 - DNA, 22 bp, synthetic construct
  source 1..22: mol_type = other DNA
tttccactttttctacctttta

SEQ ID NO: 16 - DNA, 22 bp, synthetic construct
  source 1..22: mol_type = other DNA
tttcctttttttcaacctttta

SEQ ID NO: 17 - DNA, 22 bp, synthetic construct
  source 1..22: mol_type = other DNA
tttcctttttttctacctttta

SEQ ID NO: 18 - DNA, 22 bp, synthetic construct
  source 1..22: mol_type = other DNA
tatcctttttttctacctttta

SEQ ID NO: 19 - DNA, 22 bp, synthetic construct
  source 1..22: mol_type = other DNA
tatcctttttttctagctttta

SEQ ID NO: 20 - DNA, 22 bp, synthetic construct
  source 1..22: mol_type = other DNA
tttccactttttcaaggtttta

SEQ ID NO: 21 - DNA, 22 bp, synthetic construct
  source 1..22: mol_type = other DNA
tatcctttttttctaggtttta

SEQ ID NO: 22 - DNA, 22 bp, synthetic construct
  source 1..22: mol_type = other DNA
tatcctttttttctaggtgtta

SEQ ID NO: 23 - DNA, 22 bp, synthetic construct
  source 1..22: mol_type = other DNA
tttccacttatacctcctttta

SEQ ID NO: 24 - DNA, 22 bp, synthetic construct
  source 1..22: mol_type = other DNA
tttccatctatacaacctttta

SEQ ID NO: 25 - DNA, 22 bp, synthetic construct
  source 1..22: mol_type = other DNA
tttccatctatacctcctttta

SEQ ID NO: 26 - DNA, 22 bp, synthetic construct
  source 1..22: mol_type = other DNA
tttccacttatacaacttttta

SEQ ID NO: 27 - DNA, 22 bp, synthetic construct
  source 1..22: mol_type = other DNA
tttgcacttatacaacctttta

SEQ ID NO: 28 - DNA, 22 bp, synthetic construct
  source 1..22: mol_type = other DNA
tttccatctatacctcttttta

SEQ ID NO: 29 - DNA, 22 bp, synthetic construct
  source 1..22: mol_type = other DNA
tttgcatctatacctcctttta

SEQ ID NO: 30 - DNA, 22 bp, synthetic construct
  source 1..22: mol_type = other DNA
tttgcatctatacctcttttta

SEQ ID NO: 31 - DNA, 22 bp, synthetic construct
  source 1..22: mol_type = other DNA
tctgcatctatacctcttttta

SEQ ID NO: 32 - DNA, 22 bp, synthetic construct
  source 1..22: mol_type = other DNA
tttgcatctatacctctttcta

SEQ ID NO: 33 - DNA, 22 bp, synthetic construct
  source 1..22: mol_type = other DNA
tctgcatctatacctctttcta

SEQ ID NO: 34 - DNA, 22 bp, Homo sapiens
  source 1..22: mol_type = other DNA
tatcctttttttctaggttttc

SEQ ID NO: 35 - DNA, 22 bp, Homo sapiens
  source 1..22: mol_type = other DNA
tatcctttttttctaggtttgt

SEQ ID NO: 36 - DNA, 22 bp, Mus musculus
  source 1..22: mol_type = other DNA
tgtcctttttttctaggtgttt

SEQ ID NO: 37 - DNA, 22 bp, Homo sapiens
  source 1..22: mol_type = other DNA
tctgcatctatacctctttgac

SEQ ID NO: 38 - DNA, 22 bp, Homo sapiens
  source 1..22: mol_type = other DNA
tctgcatctatacctcttttga

SEQ ID NO: 39 - DNA, 22 bp, synthetic construct
  source 1..22: mol_type = other DNA
aaacacctagaaaaaaaggata

SEQ ID NO: 40 - DNA, 22 bp, synthetic construct
  source 1..22: mol_type = other DNA
aaacacctagaaaaaaaggata

SEQ ID NO: 41 - DNA, 22 bp, synthetic construct
  source 1..22: mol_type = other DNA
tttccacttattcaacctttta

SEQ ID NO: 42 - DNA, 790 bp, synthetic construct
  source 1..790: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctccccccctccccacccccaattttgtatttatttattttttaattattttgtgcagcgatgggggcggggggggggggggcgcgcgccaggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 43 - DNA, 20 bp, synthetic construct
  source 1..20: mol_type = other DNA
ctggccgtggtcgtacatga

SEQ ID NO: 44 - DNA, 20 bp, synthetic construct
  source 1..20: mol_type = other DNA
tcaccgacatgggcaacctt

SEQ ID NO: 45 - DNA, 24 bp, synthetic construct
  source 1..24: mol_type = other DNA
gagaacgtggagcacgcgtacggc

SEQ ID NO: 46 - DNA, 24 bp, synthetic construct
  source 1..24: mol_type = other DNA
ggcccggtttgagacggtaccagc

SEQ ID NO: 47 - DNA, 20 bp, synthetic construct
  source 1..20: mol_type = other DNA
gacagcacggacacggaact

SEQ ID NO: 48 - DNA, 20 bp, synthetic construct
  source 1..20: mol_type = other DNA
tcgtccaggtcgtcgtcatc

SEQ ID NO: 49 - DNA, 20 bp, synthetic construct
  source 1..20: mol_type = other DNA
gaccgcatcagcgagagctt

SEQ ID NO: 50 - DNA, 20 bp, synthetic construct
  source 1..20: mol_type = other DNA
ctcgcagacacgactcgaac

SEQ ID NO: 51 - DNA, 20 bp, synthetic construct
  source 1..20: mol_type = other DNA
cggccatcaagaagtacgag

SEQ ID NO: 52 - DNA, 20 bp, synthetic construct
  source 1..20: mol_type = other DNA
aagtggctctggcctatgtc

SEQ ID NO: 53 - DNA, 18 bp, synthetic construct
  source 1..18: mol_type = other DNA
ggcgttcgcagatttgga

SEQ ID NO: 54 - DNA, 20 bp, synthetic construct
  source 1..20: mol_type = other DNA
tcccaggtgagcagcagtct

SEQ ID NO: 55 - DNA, 23 bp, synthetic construct
  source 1..23: mol_type = other DNA
acctgaaggctctgcgcggactc

SEQ ID NO: 56 - DNA, 20 bp, synthetic construct
  source 1..20: mol_type = other DNA
ccgcccggaaagagattatt

SEQ ID NO: 57 - DNA, 21 bp, synthetic construct
  source 1..21: mol_type = other DNA
cggagttcagattggtcagtt

SEQ ID NO: 58 - DNA, 26 bp, synthetic construct
  source 1..26: mol_type = other DNA
nagctgctggatcagattgccaagan

SEQ ID NO: 59 - DNA, 22 bp, synthetic construct
  source 1..22: mol_type = other DNA
tgccgtgtttatctcgtcaact

SEQ ID NO: 60 - DNA, 25 bp, synthetic construct
  source 1..25: mol_type = other DNA
cccgccatgctacttatctacttaa

SEQ ID NO: 61 - DNA, 16 bp, synthetic construct
  source 1..16: mol_type = other DNA
nttggcgagatttttn

SEQ ID NO: 62 - DNA, 19 bp, synthetic construct
  source 1..19: mol_type = other DNA
tggacagcctgagcgagaa

SEQ ID NO: 63 - DNA, 22 bp, synthetic construct
  source 1..22: mol_type = other DNA
gcagagacagaggagcaatgtg

SEQ ID NO: 64 - DNA, 30 bp, synthetic construct
  source 1..30: mol_type = other DNA
ncggccggtgattcctctgtttctaattcn

(19) United States
(12) Patent Application Publication
(10) Pub. No.: US 2024/0398984 A1
(43) Pub. Date: Dec. 5, 2024

(54) TREATMENT OF HSV-2 USING A MEGANUCLEASE

(71) Applicant: Fred Hutchinson Cancer Center, Seattle, WA (US)

(72) Inventors: Keith R. Jerome, Seattle, WA (US); Barry L. Stoddard, Seattle, WA (US); Abigail R. Lambert, Seattle, WA (US)

(73) Assignee: Fred Hutchinson Cancer Center, Seattle, WA (US)

(21) Appl. No.: 18/670,419

(22) Filed: May 21, 2024

Related U.S. Application Data
(60) Provisional application No. 63/503,541, filed on May 22, 2023.

(51) Int. Cl. A61K 48/00; A61K 9/00; A61K 38/46; A61P 31/22; C12N 5/0793; C12N 15/86

(52) U.S. Cl. CPC A61K 48/005; A61K 9/0019; A61K 38/465; A61K 48/0075; A61P 31/22; C12N 5/062; C12N 15/86; C12N 2750/14143

(57) ABSTRACT

Described herein are compositions and methods for reducing or eliminating latent herpes simplex virus type 2 (HSV-2) from an HSV-2-infected cell, or for reducing or eliminating latent HSV-2 reactivation in an HSV-2-infected cell, leading to a viable curative approach for latent HSV-2 infection. The composition comprises a plurality of viral vectors, wherein the viral vectors comprise a sequence encoding an HSV-2-specific meganuclease. The method comprises delivering to an HSV-2-infected cell a plurality of one or more viral vectors, wherein each of the one or more viral vectors includes a sequence encoding an HSV-2-specific meganuclease.

### CROSS-REFERENCE TO RELATED APPLICATION

This application claims the benefit of U.S. Provisional Patent Application No. 63/503,541, filed May 22, 2023, the disclosure of which is hereby incorporated by reference in its entirety.

### STATEMENT OF GOVERNMENT LICENSE RIGHTS

This invention was made with Government support under GM105691 and AI132599 awarded by National Institutes of Health. The Government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 1896-P83US_Seq_List_20240521.xml. The XML file is 61,447 bytes; was created on May 21, 2024; and is being submitted electronically via Patent Center with the filing of the specification.

### BACKGROUND

Herpes simplex virus type 2 (HSV-2) is a member of the Herpesviridae family of double-stranded DNA viruses. HSV-2 is a widespread pathogen and is the leading cause of genital herpes, a sexually transmitted disease. It is estimated that in 2016, 491.5 million people worldwide aged 15 to 49 years were infected with HSV-2, corresponding to 13% of the world's population for this age range. HSV-2 is a lifelong disease, with prevalence increasing with age, and women approximately twice as susceptible to infection compared with men.

HSV-2 is transmitted through contact of body surfaces with the unprotected genital areas of an infected person, most often through sexual contact. HSV-2 infection often leads to recurring lesions on the genitals and perianal regions, as well as viral shedding from mucosal surfaces. Viral shedding can also occur in the absence of lesions or other physical symptoms.

Subsequent to primary infection of the skin or mucosal surfaces, HSV-2 can migrate from the site of primary infection to infect neuronal nuclei such as sensory (e.g. dorsal root) and autonomic (e.g. superior cervical and major pelvic) ganglia of the peripheral nervous system through retrograde transport, establishing latent infection. Such neuronal infection can occur without evident cytopathology.

HSV-2 lifelong infection exists primarily in the latent state in neurons. However, HSV-2 can subsequently reactivate from latency by certain factors, including stress, whereupon it migrates back through axonal retrograde transport to the primary infection location or other peripheral sites, causing new lesions and/or virus shedding.

HSV-2 lesions are often painful, are stigmatizing and have social consequences affecting sexual relationships, and can lead to additional serious health risks. For example, in addition to lesions, HSV-2 infection can cause meningoencephalitis (brain infection) and disseminated infection. Additionally, HSV-2 infection leads to a three-fold increased risk for acquiring and spreading human immunodeficiency virus (HIV). HSV-2 can also be transmitted to neonates during childbirth. HSV-2 infections in immunocompromised individuals or neonates can be fatal.

Currently, no cure exists for an HSV-2 infection. Treatment options include antiviral medications such as acyclovir, famciclovir, and valacyclovir. While these medications can reduce the severity and frequency of symptoms, they cannot cure the infection, or reduce, eliminate, or prevent the latent virus that drives recurrent disease. In addition, use of antiviral medications requires patients to recognize HSV-2 infection symptoms, seek medical diagnosis, have access to a pharmacy and funds to pay for ongoing treatment, and comply with the treatment regimen. Such requirements may be untenable for individuals in regions of the world lacking access to continued antiviral treatment, or individuals unaware of continued infection and transmissibility during latency periods.

Vaccines have shown some promise in animal models. However, the ability of vaccines to induce immunity within the nervous system, as would be required to intercept the virus before neuronal infection or during the reactivation process, remains unproven. Vaccines have thus far been ineffective in treating the millions of people already infected with HSV-2.

Therefore, a need exists for novel and effective therapeutic treatment of HSV-2 and latent HSV-2 infection. The present disclosure addresses these needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Disclosed herein is a composition comprising a plurality of one or more viral vectors, wherein each of the one or more viral vectors includes a sequence encoding an HSV-2-specific meganuclease.

Also disclosed herein is a method of reducing or eliminating latent HSV-2 or HSV-2 reactivation in a cell, comprising administering to a cell a plurality of one or more viral vectors including a sequence encoding an HSV-2-specific meganuclease.

Additionally disclosed herein is a method of reducing or eliminating latent HSV-2 or HSV-2 reactivation in a subject, comprising administering to the subject a plurality of one or more viral vectors including a sequence encoding an HSV-2-specific meganuclease.

### DESCRIPTION OF THE DRAWINGS

This patent or application file contains at least one drawing executed in color. Copies of this patent or patent application publication with color drawing(s) will be provided by the Office upon request and payment of the necessary fee.

The foregoing aspects and many of the attendant advantages of this invention will become more readily appreciated as the same become better understood by reference to the following detailed description, when taken in conjunction with the accompanying drawings, wherein:

FIG. 1 shows the series of libraries designed for engineering the thermostabilized I-Onul meganuclease toward the OnuHSV2a DNA target sequence. The desired final OnuHSV2a target sequence is aligned to the wildtype I-Onul sequence, and a series of libraries is designed to make small changes to the meganuclease in a step-wise manner, starting at the center of the target sequence (nearest the enzyme active site) and moving progressively outward. Positions in the DNA target sequences which differ from the wildtype I-Onul recognition sequence are depicted as lower-case letters. The structure of the I-Onul meganuclease is shown above the target sequence alignment, with the mismatched positions of the bound DNA target shown red, and the corresponding positions of incorporated amino acid sidechain variation designated as the spheres.

FIG. 2 shows the series of libraries designed for engineering the thermostabilized I-Onul meganuclease toward the OnuHSV2b DNA target sequence. The desired final OnuHSV2b target sequence is aligned to the wildtype I-Onul sequence, and a series of libraries is designed to make small changes to the meganuclease in a step-wise manner, starting at the center of the target sequence (nearest the enzyme active site) and moving progressively outward. Positions in the DNA sequences which differ from the wildtype I-Onul recognition sequence are depicted as lower-case letters. The structure of the I-Onul meganuclease is shown above the target sequence alignment, with the mismatched positions of the bound DNA target shown red, and the corresponding positions of incorporated amino acid sidechain variation designated as spheres.

FIG. 3A shows an expression diagram of meganucleases on the surface of yeast. The pETCON yeast surface display vector expresses the meganuclease coding sequence with an HA epitope tag on the N-terminus of the protein and a Myc epitope tag on the C-terminus.

FIG. 3B shows a diagram of the tethered flow cytometric DNA cleavage assay. The HA tag on the surface-expressed meganuclease is stained with an anti-HA-biotin antibody and the Myc tag is stained with a fluorescent anti-Myc-FITC antibody. FITC signal can be measured to confirm full-length protein expression on the surface of the yeast. A double-stranded DNA target substrate is generated with a biotin at one end and a fluorescent A647 tag on the opposite end. The DNA substrate is pre-conjugated with streptavidin-PE (SAV-PE) and when mixed with the pre-stained meganuclease will form a biotin-streptavidin bridge between the DNA target substrate and the N-term of the meganuclease. Incubation of the meganuclease with the tethered DNA target substrate in the presence of calcium divalent metal allows the meganuclease to bind the DNA but not cleave it, as shown at the bottom left of FIG. 3B. Incubation in the presence of magnesium divalent metal allows for cleavage of the tethered DNA target substrate, as shown at the bottom right of FIG. 3B. If cleavage occurs, the A647 fluorophore on the opposite end of the DNA target substrate is released and washed away.

FIG. 3C shows that tethering of the DNA target substrate in the presence of calcium creates a sharp diagonal line on a flow cytometric plot of A647 signal vs. PE signal (upper graph upper linear cluster of dots). Cleavage of the tethered substrate in the presence of magnesium results in a loss of the A647 tag and a population of cells which drop down from the diagonal (upper graph lower broader cluster of dots). A sort gate (lower graph) can be drawn to collect the cells that demonstrate this drop in A647 signal.

FIGS. 4A-4C show DNA cleavage activity in the tethered flow cytometric cleavage assay of the final sorted populations after each of the intermediate libraries generated toward the OnuHSV2a target site. In some cases, more than one version of a library was made (example 03a, 03b) to test a wider range of variation than can fit into a single library. A647 signal from the tethered DNA substrate is plotted on the y-axis (collected with the APC channel of the cytometer) with PE signal from the N-terminal stain on the x-axis. Surface-expressed meganuclease was incubated at 37° C. in pH 7.0 buffer for 30 minutes.

FIGS. 5A-5E show DNA cleavage activity in the tethered flow cytometric cleavage assay of the final sorted populations after each of the intermediate libraries generated toward the OnuHSV2b target site. In some cases, more than one version of a library is made (example 06a, 06b) in order to test a wider range of variation than can fit into a single library. The first library (Lib01) was tested with both the wildtype I-Onul active site and the E178D active site mutation, and the E178D version of the active site was found to be superior. The E178D active site was therefore used in all subsequent libraries for this target. A647 signal from the tethered DNA substrate is plotted on the y-axis (collected with the APC channel of the cytometer) with PE signal from the N-terminal stain on the x-axis. Surface-expressed meganuclease was incubated at 37° C. in pH 7.0 buffer for 30 minutes.

FIG. 6 shows the non-tethered DNA cleavage activity assay schematic and an example gel demonstrating different levels of cleavage activity in the assay. Surface-expressed meganuclease is released from the yeast surface with the addition of DTT (disrupts the disulfide bond between the AGA1 yeast surface protein and the AGA2 fusion protein added to the meganuclease by the pETCON vector). The A647-labeled DNA target substrate is free-floating in solution with no physical tethering. The meganuclease must therefore successfully bind the DNA before it can cleave the target. The reaction is incubated in the presence of calcium divalent metal to allow for binding but no cleavage (control lane for uncut DNA target substrate). Incubation in the presence of magnesium divalent metal allows for cleavage to occur and results in a shorter cut band after sample electrophoresis on an acrylamide gel. Bands are visualized through the fluorescent signal from the A647 tag on the DNA using a Typhoon imager.

FIGS. 7A-7D shows DNA cleavage activity in the tethered flow cytometric cleavage assay of the 6 final candidates for the OnuHSV2a target. Each of the final engineered meganucleases were assayed vs. the desired OnuHSV2a target and each of two “2-off” targets identified in the human genome. A647 signal from the tethered DNA substrate is plotted on the y-axis (collected with the APC channel of the cytometer) with PE signal from the N-terminal stain on the x-axis. Surface-expressed meganuclease was incubated at 37° C. in pH 7.4 buffer for 30 minutes.

FIG. 8 shows DNA cleavage activity in the non-tethered activity assay of the 6 final candidates for the OnuHSV2a target. Each of the final engineered meganucleases were assayed vs. the desired OnuHSV2a target, two “2-off” targets identified in the human genome, and a single “1-off” target identified in the mouse genome. The reactions were incubated at 37° C. in pH 7.4 buffer for 30 minutes.

FIGS. 9A-9D shows DNA cleavage activity in the tethered flow cytometric cleavage assay of the 6 final candidates for the OnuHSV2b target. Each of the final engineered meganucleases were assayed vs. the desired OnuHSV2b target and each of two “3-off” targets identified in the human genome. A647 signal from the tethered DNA substrate is plotted on the y-axis (collected with the APC channel of the cytometer) with PE signal from the N-terminal stain on the x-axis. Surface-expressed meganuclease was incubated at 37° C. in pH 7.4 buffer for 30 minutes.

FIG. 10 shows DNA cleavage activity in the non-tethered activity assay of the 6 final candidates for the OnuHSV2b target. Each of the final engineered meganucleases were assayed vs. the desired OnuHSV2b target (Des) and two different “3-off” targets identified in the human genome (A and B). The same gels were imaged after three progressively longer exposure times in an effort to see any faint bands present (shown as a vertical stack of three band images labeled “Normal exposure” for the shortest gel exposure time up to “Darkest Exposure” for the longest exposure time.) The reactions were incubated at 37° C. in pH 7.4 buffer for 30 minutes.

FIG. 11A shows a detailed schematic of the plasmid for a mammalian reporter system, which comprises two sites for HSV-2 Onu, along with sites for m4 and CRISPR/Cas9 (LAT-gRNA1 and LATgRNR2). M4 is a control meganuclease that does not mediate anti-HSV-2 activity. The reporter plasmid, along with a plasmid expressing the candidate meganuclease or CRISP/Cas9, is co-transfected into mammalian 293T cells. With a functioning enzyme, the sequence between the target sites is removed, and the resulting PCR amplicon is smaller than it is in the uncleaved state. FIGS. 11B and 11C show gels which illustrate enzyme cleavage activity as demonstrated by the smaller PCR amplicon (lower band/shorter bp sequence), in comparison with the uncleaved PCR amplicon (higher band/longer bp sequence). A single higher/longer base pair band indicates no activity (e.g. left 2 lane controls). All experiments were conducted in duplicate. The spCas9 “duplicates” were conducted with two different gRNAs. Quantitation was performed by dosimetry to generate the bar graphs at the bottom of the panels B and C. All meganuclease candidates tested worked, as evidenced by the smaller PCR amplicon band (lower band/shorter bp sequence) in the gel. Sequences 1831 and 1832 appear to have the strongest activity, as evidenced by the highest percent deletion (31% and 37%, respectively). Negative controls (wild type Onu and sequence 1840) were negative as expected.

FIG. 12 shows an alternative approach to confirming the enzyme's activity through Sanger sequencing of amplicons. Cleavage of the predicted site (shown at the top with the 3-segment line indicating the staggered ends of the cut), is demonstrated by the tracing changing from “clean” with relatively little background (small peaks under the mail tracing), to “dirty” with lots of subdominant peaks as indicated by the arrows. Cleavage is most dramatic with 1831 and 1832, wherein numerous subdominant peaks appeared, demonstrating significant activity by these variants.

FIG. 13 shows the weights of animals receiving the indicated enzyme delivered by AAV9 under control of the CbH promoter. Unlike animals that receive m4 under the same conditions (not shown), animals receiving the Onu-based enzymes gain weight normally after therapy, suggesting the Onu-based enzymes are better tolerated with no overt signs of toxicity.

FIGS. 14A through 14C show liver IFC, TG axonopathy, and TG inflammation in mice that received the indicated meganuclease enzyme delivered by AAV9 under control of the CbH promoter. Unlike animals which received m4 under the same conditions (not shown), no detectable liver toxicity or neurotoxicity was observed.

FIGS. 15A through 15D show HSV virus shedding after JQ1 induced shedding in controls or animals receiving the indicated meganuclease enzyme delivered by AAV9 under control of the CbH promoter. As shown, 6 of 10 control animals shed HSV at the indicated levels after JQ1; only 3/9 animals shed when treated with 1838; only a single animal shed after treatment with 1834; only a single animal shed after treatment with 1831.

### DETAILED DESCRIPTION

This application describes compositions and methods for reducing or eliminating latent herpes simplex virus type 2 (HSV-2) from an HSV-2-infected cell, or for reducing or eliminating latent HSV-2 reactivation in an HSV-2-infected cell, leading to a viable curative approach for latent HSV-2 infection.

Unless specifically defined herein, all terms used herein have the same meaning as they would to one skilled in the art.

In one aspect, this disclosure provides a composition comprising a plurality of one or more viral vectors, wherein each of the one or more viral vectors includes a sequence encoding an HSV-2-specific meganuclease.

As used herein, “includes” means the sequence accompanies the viral vector whether the sequence is contained within the viral vector, has been excreted by the viral vector, is bound to the viral vector, or is otherwise associated with the viral vector.

In some embodiments, this disclosure provides a composition for reducing or eliminating latent HSV-2 or HSV-2 reactivation in a cell, comprising a plurality of one or more viral vectors, wherein each of the one or more viral vectors includes a sequence encoding an HSV-2-specific meganuclease.

As used herein, “viral vectors” are vectors used to deliver or administer compositions to cells, wherein such compositions comprise genetic material such as deoxyribonucleic acid (DNA) and/or ribonucleic acid (RNA). “Viral vectors” refers to the use of adeno-associated virus (AAV) vectors or any viral vector engineered from an AAV. For example, the viral vector of the one or more viral vectors comprises self-complementary adeno-associated viruses (scAAVs), single-stranded adeno-associated viruses (ssAAVs), and known serotypes of the AAV vectors, the scAAV vectors, and the ssAAV vectors.

In some embodiments, the one or more viral vector comprises one viral vector. In some embodiments, the one or more viral vector comprises two viral vectors. In some embodiments, the one or more viral vector comprises three viral vectors.

In some embodiments, each of the one or more viral vectors is an AAV. The AAV can comprise any serotype well known to those having ordinary skill in the art. For example, the one or more AAVs can comprise AAV1, AAV2, AAV3, AAV4, AAV5, AAV6, AAV7, AAV8, AAV9, AAV10, AAV11, AAV12, AAV-Dj, AAV-Dj/8, PHP.eB, PHP.S, AAV-rh8, AAV-rh10 serotype adeno-associated virus, or a combination thereof. In some embodiments, the one or more AAVs comprises AAV-rh10, AAV8, AAV9, AAV1, AAV-Dj/8 serotype adeno-associated virus, or a combination thereof.

In some embodiments, the one or more viral vector is a self-complementary adeno-associated virus (scAAV), a single-stranded adeno-associated virus (ssAAV), or a combination thereof.

In some embodiments, the more than one viral vector comprises more than one scAAV; more than one ssAAV; or a combination of one or more each of scAAV and SSAAV.

In some embodiments, the viral vector comprises both a scAAV and a ssAAV. For example, in some embodiments, the viral vector comprises a plurality of scAAVs and a plurality of ssAAVs. In such embodiments, the ratio of scAAV to ssAAV can be between about 1:99 and about 99:1; between about 10:90 and about 90:10; between about 20:80 and about 80:20; between about 30:70 and about 70:30; between about 50:50 and about 99:1; about 10:90; about 20:80; about 30:70: about 40:60; about 50:50; about 60:40; about 70:30; about 80:20; about 90:10; or about 99:1.

In some embodiments, the viral vector is a ssAAV. In some embodiments, the viral vector comprises a plurality of ssAAVs. In some embodiments, the ssAAV comprises one ssAAV serotype. In other embodiments, the ssAAV comprises more than one ssAAV serotype, two ssAAV serotypes, three ssAAV serotypes, four ssAAV serotypes, five ssAAV serotypes, or six ssAAV serotypes.

In some embodiments, the ssAAV comprises any serotype known to those having ordinary skill in the art. For example, in some embodiments, the one or more ssAAV comprises ssAAV1, ssAAV2, ssAAV3, ssAAV4, ssAAV5, ssAAV6, ssAAV7, ssAAV8, SSAAV9, ssAAV10, ssAAV11, ssAAV12, ssAAV-Dj, ssAAV-Dj/8, PHP.eB, PHP.S, ssAAV-rh8, ssAAV-rh10 serotype adeno-associated virus, or a combination thereof. In some embodiments, the one or more ssAAV comprises ssAAV9, ssAAV-Dj/8, ssAAV-rh10, ssAAV8, ssAAV1, another serotype adeno-associated virus, or a combination thereof. In some embodiments, the one or more ssAAV comprises ssAAV9, ssAAV-Dj/8, ssAAV-rh10, ssAAV8, ssAAV1, or a combination thereof. In some embodiments, the one or more ssAAV comprises ssAAV9. In some embodiments, the one or more ssAAV comprises ssAAV-Dj/8. In some embodiments, the one or more ssAAV comprises ssAAV-rh10. In some embodiments, the one or more ssAAV comprises ssAAV8. In some embodiments, the one or more ssAAV comprises ssAAV1.

In some embodiments, the viral vector is a scAAV. In some embodiments, the viral vector comprises a plurality of scAAVs. In some embodiments, the scAAV comprises one scAAV serotype. In other embodiments, the scAAV comprises more than one scAAV serotype, two scAAV serotypes, three scAAV serotypes, four scAAV serotypes, five scAAV serotypes, or six scAAV serotypes.

The scAAV comprises any serotype known to those having ordinary skill in the art. For example, in some embodiments, the one or more scAAV comprises scAAV1, scAAV2, scAAV3, scAAV4, scAAV5, scAAV6, scAAV7, scAAV8, scAAV9, scAAV10, scAAV11, scAAV12, scAAV-Dj, scAAV-Dj/8, PHP.eB, PHP.S, ssAAV-rh8, ssAAV-rh10 serotype adeno-associated virus, or a combination thereof. In some embodiments, the one or more scAAV comprises scAAV9, scAAV-Dj/8, scAAV-rh10, scAAV8, scAAV1, another serotype adeno-associated virus, or a combination thereof. In some embodiments, the one or more scAAV comprises scAAV9, scAAV-Dj/8, scAAV-rh10, scAAV8, scAAV1, or a combination thereof. In some embodiments, the one or more scAAV comprises scAAV9. In some embodiments, the one or more scAAV comprises scAAV-Dj/8. In some embodiments, the one or more scAAV comprises scAAV-rh10. In some embodiments, the one or more scAAV comprises scAAV8. In some embodiments, the one or more scAAV comprises scAAV1.

In some embodiments, the more than one viral vector comprises a combination of different adeno-associated viruses and serotypes of an adeno-associated virus.

“Sequences” as used herein comprise DNA strands and/or RNA strands. DNA strands comprise the natural DNA bases cytosine (C), guanine (G), adenine (A), and/or thymine (T). RNA strands comprise the natural RNA bases cytosine (C), guanine (G), adenine (A), and/or uracil (U). In some embodiments, the sequence is a double strand or single strand of DNA. In other embodiments, the sequence is a strand of RNA.

In some embodiments, the viral vector comprises a DNA sequence, wherein the DNA sequence encodes an HSV-2 specific meganuclease. In some embodiments, the viral vector comprises a different DNA sequence, wherein the different DNA sequence encodes the HSV-2 specific meganuclease. Due to the degeneracy of the DNA code, some embodiments may use DNA sequences optimized to reduce the use of rare codons, to reduce the frequency of CpG motifs, or other choices to optimize production of the meganuclease.

In some embodiments, each of the one or more viral vectors comprises the same DNA sequence encoding an HSV-2-specific meganuclease. In some embodiments, each of the one or more viral vectors comprises a DNA sequence encoding the same HSV-2-specific meganuclease. In some embodiments, each of the one or more viral vectors comprises a different DNA sequence encoding an HSV-2-specific meganuclease. In some embodiments, each of the one or more viral vectors comprises a DNA sequence encoding a different HSV-2-specific meganuclease. In some embodiments, some of the one or more viral vectors comprise the same DNA sequence encoding an HSV-2-specific meganuclease, and some of the one or more viral vectors comprise a different DNA sequence encoding an HSV-2-specific meganuclease. In some embodiments, some of the one or more viral vectors comprise a DNA sequence encoding the same HSV-2-specific meganuclease, and some of the one or more viral vectors comprise a DNA sequence encoding a different HSV-2-specific meganuclease.

In some embodiments, the sequence encoding the HSV-2-specific meganuclease is configured to encode an HSV-2-specific meganuclease which targets one or more HSV-2 genes essential for replication.

In some embodiments, the sequence encoding the HSV-2-specific meganuclease is configured to encode an HSV-2-specific meganuclease which induces one or more DNA double strand breaks.

A “meganuclease” is an enzyme which recognizes and cleaves DNA, such as double-stranded DNA. A meganuclease is also known as an endonuclease or homing endonuclease.

As used herein, a meganuclease is an endonuclease having a polynucleotide recognition site of at least 12 base pairs (bp); from 12 bp to 60 bp; from 12 bp to 40 bp; from 15 to 60 bp; from 15 to 40 bp; from 15 to 30 bp; from 18 to 26 bp; or from 20-24 bp. In some embodiments, the meganuclease is an endonuclease having a polynucleotide recognition site of 22 base pairs.

The meganuclease can be either monomeric or dimeric. The meganuclease can be any natural meganuclease such as a homing endonuclease. In some embodiments, the meganuclease is an engineered, artificial, or synthetic meganuclease, such as a meganuclease derived from the homing endonuclease group I introns and inteins; Zinc-finger proteins; group II intron proteins; or chemically-modified nucleic acid derivatives. The meganuclease can be any engineered meganuclease, such as a meganuclease engineered by translation from an RNA encoded by a synthetic DNA sequence. In some embodiments, the meganuclease is post-translationally-modified.

I-Onul is an intron-encoded homing endonuclease, and is a “meganuclease.” Such meganuclease is an enzyme which recognizes and cleaves both strands of an, e.g., 22 base pair DNA target, leading to the generation of a DNA double strand break. I-Onul was altered through protein engineering approaches to recognize altered DNA target sequences of the same length, and then used to disrupt or otherwise modify genomic targets that harbor the corresponding target sequence.

In some embodiments, the meganuclease comprises an engineered variant derived from thermostabilized I-Onul (eOnuTherm). In some embodiments, the meganuclease comprises the sequence of any one of the sequences identified as OnuHSV2a_1831, OnuHSV2a_1832, OnuHSV2a_1834, OnuHSV2a_1838, OnuHSV2a_1840, and OnuHSV2a_1856 (identified herein as SEQ ID NOs: 1-6, respectively). In some embodiments, the meganuclease comprises the sequence identified as OnuHSV2a_1831. In some embodiments, the meganuclease comprises the sequence identified as OnuHSV2a_1832.

In some embodiments, the meganuclease comprises one or more of the following sequences:

OnuHSV2a_1831:
(SEQ ID NO: 1)
SINPWILTGFADAEGSFMLRIRKRNKMSVGYLTELVFGITLHNKDKSILE
NIQSTWGVGVIYNSGDNAVSLRVTRFEDLKVIIDHFEKYPLITQKYADYM
LFKQAFNVMENKEHLTIEGIKELVRIKAKLNWGLTDELKKAFPEIISKER
SLINKNIPNFKWLAGFTSGEGCFFVALRKAKSKRGVQVYLRFSIGQHIRD
KNLMNSLITYLGCGYIEKHNKSEFSWLEFRVYKFSDIRDKIIPFFQEYTL
IGTKLKDFEDWCKVAKLIEEKKHLTEEGLDEIKKIKLNMNKGRVF.
OnuHSV2a_1832:
(SEQ ID NO: 2)
SINPWILTGFADAEGSFMLRIRKRNKMSVGYLTELVFGITLHNKDKSILE
NIQSTWGVGVIYNSGDNAVSLRVTRFEDLKVIIDHFEKYPLITQKYADYM
LFKQAFNVMENKEHLTIEGIKELVRIKARLNWGLTDELKKAFPEIISKER
SLINKNIPNFKWLAGFTSGEGCFFVALRKTKFKLGVQVYLRFSIGQHIRD
KNLMNSLITYLGCGYIEKHNKSEFSWLEFRVYKFSDIRDKIIPFFQEYTL
IGTKLKDFEDWCKVAKLIEEKKHLTEEGLDEIKKIKLNMNKGRVF.
OnuHSV2a_1834:
(SEQ ID NO: 3)
SINPWILTGFADAEGSFMLRIRKRNKMSVGYLTELVFGITLHNKDKSILE
NIQSTWGVGVIYNSGDNAVSLRVTRFEDLKVIIDHFEKYPLITQKYADYM
LFKQAFNVMENKEHLTIEGIKELVRIKAKLNWGLTDELKKAFPEIISKER
SLINKNIPNFKWLAGFTSGEGCFFVALRKNKHKSGVQVYLRFSIGQHIRD
KNLMNSLITYLGCGYIEKHNKSEFSWLEFRVYKFSDIRDKIIPFFQEYTL
IGTKLKDFEDWCKVAKLIEEKKHLTEEGLDEIKKIKLNMNKGRVF.
OnuHSV2a_1838:
(SEQ ID NO: 4)
SINPWILTGFADAEGSFMLRIRKRNKMSVGYLTELVFGITLHNKDKSILE
NIQSTWGVGVIYNSGDNAVSLRVTRFEDLKVIIDHFEKYPLITQKYADYM
LFKQAFNVMENKEHLTIEGIKELVRIKAKLNWGLTDELKKAFPEIISKER
SLINKNIPNFKWLAGFTSGEGCFFVALRKRKSKDGVQVYLRFSIGQHIRD
KNLMNSLITYLGCGYIEKHNKSEFSWLEFRVYKFSDIRDKIIPFFQEYTL
IGTKLKDFEDWCKVAKLIEEKKHLTEEGLDEIKKIKLNMNKGRVF.
OnuHSV2a_1840:
(SEQ ID NO: 5)
SINPWILTGFADAEGSFMLRIRKRNKMSVGYLTELVFGITLHNKDKSILE
NIQSTWGVGVIYNSGDNAVSLRVTRFEDLKVIIDHFEKYPLITQKYADYM
LFKQAFNVMENKEHLTIEGIKELVRIKAKLNWGLTDELKKAFPEIISKER
SLINKNIPNFKWLAGFTSGEGCFFVALRKSKRKIGVQVYLRFSIGQHIRD
KNLMNSLITYLGCGYIEKHNKSEFSWLEFRVYKFSDIRDKIIPFFQEYTL
IGTKLKDFEDWCKVAKLIEEKKHLTEEGLDEIKKIKLNMNKGRVF.
OnuHSV2a_1856:
(SEQ ID NO: 6)
SINPWILTGFADAEGSFMLRIRKRNKMSVGYLTELVFGITLHNKDKSILE
NIQSTWGVGVIYNSGDNAVSLRVTRFEDLKVIIDHFEKYPLITQKYADYM
LFKQAFNVMENKEHLTIEGIKELVRIKAKLNWGLTDELKKAFPEIISKER
SLINKNIPNFKWLAGFTSGEGCFFVALRKSKQKMGVQVYLRFSIGQHIRD
KNLMNSLITYLGCGYIEKHNKSEFSWLEFRVYKFSDIRDKIIPFFQEYTL
IGTKLKDFEDWCKVAKLIEEKKHLTEEGLDEIKKIKLNMNKGRVF.

In some embodiments, the meganuclease comprises the sequence of any one of the sequences identified as OnuHSV2b_2269. OnuHSV2b_2275. OnuHSV2b_2279. OnuHSV2b_2287. OnuHSV2b_2288. and OnuHSV2b_2290 (identified herein as SEQ ID NOs: 7-12).

In some embodiments, the meganuclease comprises one or more of the following sequences:

OnuHSV2b_2269:
(SEQ ID NO: 7)
SINPWILTGFADAEGCFLLRIFKDNKMSVGYGTRLVFQIWLHNKDKSILE
NIQSTWGVGVIANFGDNAVSLRVSRFEDLKVIIDHFEKYPLITQKYADYM
LFKQAFNVMENKEHLTIEGIKELVRIKAKLNWGLTDELKKAFPEIISKER
SLINKNIPNFKWLAGFTSGDGCFYVNLKKQKGKLGVGVQLVFDITQHIRD
KNLMNSLITYLGCGYINKFNKSEFSILNFRVTKFSDIRDKIIPFFQEYTL
IGTKLKDFEDWCKVAKLIEEKKHLTEEGLDEIKKIKLNMNKGRVF.
OnuHSV2b_2275:
(SEQ ID NO: 8)
SINPWILTGFADAEGCFLLRIFKDNKMSVGYGTRLVFQIWLHNKDKSILE
NIQSTWGVGVIANFGDNAVSLRVSRFEDLKVIIDHFEKYPLITQKYADYM
LFKQAFNVMENKEHLTIEGIKELVRIKAKLNWGLTDELKKAFPEIISKER
SLINKNIPNFKWLAGFTSGDGCFYVNLKKDKVKGGVGVQLVFDITQHIRD
KNLMNSLITYLGCGYINKSNKSEFSVLNFRVTKFSDIRDKIIPFFQEYTL
IGTKLKDFEDWCKVAKLIEEKKHLTEEGLDEIKKIKLNMNKGRVF.
OnuHSV2b_2279:
(SEQ ID NO: 9)
SINPWILTGFADAEGCFLLRIFKDNKMSVGYGTRLVFQIWLHNKDKSILE
NIQSTWGVGVIANFGDNAVSLRVSRFEDLKVIIDHFEKYPLITQKYADYM
LFKQAFNVMENKEHLTIEGIKELVRIKAKLNWGLTDELKKAFPEIISKER
SLINKNIPNFKWLAGFTSGDGCFYVNLKKQKSKLGLGVQLVFDITQHIRD
KNLMNSLITYLGCGYINKFNKSEFSTLNFRVTKFSDIRDKIIPFFQEYTL
IGTKLKDFEDWCKVAKLIEEKKHLTEEGLDEIKKIKLNMNKGRVF.
OnuHSV2b_2287:
(SEQ ID NO: 10)
SINPWILTGFADAEGCFLLRIFKNNKMSVGYGTRLVFQIWLHNKDKSILE
NIQSTWGVGVIANFGDNAVSLRVSRFEDLKVIIDHFEKYPLITQKYADYM
LFKQAFNVMENKEHLTIEGIKELVRIKAKLNWGLTDELKKAFPEIISKER
SLINKNIPNFKWLAGFTSGDGCFYVNLKKQKSKWGVGVQLVFDITQHIRD
KNLMNSLITYLGCGYINKSNKSEFSTLNFRVTKFSDIRDKIIPFFQEYTL
IGTKLKDFEDWCKVAKLIEEKKHLTEEGLDEIKKIKLNMNKGRVF.
OnuHSV2b_2288:
(SEQ ID NO: 11)
SINPWILTGFADAEGCFLLRIYKDNKMSVGYGTRLVFQIWLHNKDKSILE
NIQSTWGVGVIANFGDNAVSLRVSRFEDLKVIIDHFEKYPLITQKYADYM
LFKQAFNVMENKEHLTIEGIKELVRIKAKLNWGLTDELKKAFPEIISKER
SLINKNIPNFKWLAGFTSGDGCFYVNLIKQKTKLGLRVQLVFDITQHIRD
KNLMNSLITYLGCGYINKSNKSEFSVLNFRVTKFSDIRDKIIPFFQEYTL
IGTKLKDFEDWCKVAKLIEEKKHLTEEGLDEIKKIKLNMNKGRVF.
OnuHSV2b_2290:
(SEQ ID NO: 12)
SINPWILTGFADAEGCFLLRIFKNNKMSVGYGTRLVFQIWLHNKDKSILE
NIQSTWGVGVIANFGDNAVSLRVSRFEDLKVIIDHFEKYPLITQKYADYM
LFKQAFNVMENKEHLTIEGIKELVRIKAKLNWGLTDELKKAFPEIISKER
SLINKNIPNFKWLAGFTSGDGCFYVNLKKEKGKWGLHVQLVFDITQHIRD
KNLMNSLITYLGCGYINKSNKSEFSTLHFRVTKFSDIRDKIIPFFQEYTL
IGTKLKDFEDWCKVAKLIEEKKHLTEEGLDEIKKIKLNMNKGRVF.

Gene editing using meganucleases directly targets latent genomes through disrupting or eliminating HSV-2 while preserving neurons, thus eliminating the possibility of viral reactivation and pathogenesis. Specifically, gene editing by meganuclease proteins comprises introducing the meganuclease into ganglia by adeno-associated virus (AAV) vectors. Once in the neurons, the meganuclease bound and cleaved the viral DNA strand, disrupting the HSV-2 viral genome. At least two independent cleavage events were optimal to negate viral DNA ligation and repair mechanisms. Once the viral genome was disrupted, the virus was incapable of replicating, and eventually the latent virus was eliminated from the neuron.

In some embodiments, the HSV-2-specific meganuclease is configured to induce one or more DNA double strand breaks. In some embodiments, the HSV-2-specific meganuclease is configured to induce one DNA double strand break. In some embodiments, the HSV-2-specific meganuclease is configured to induce two DNA double strand breaks. In some embodiments, the HSV-2-specific meganuclease is configured to induce three DNA double strand breaks.

In some embodiments, each of the one or more HSV-2-specific meganucleases is configured to induce one or more DNA double strand breaks of an HSV-2 gene. In some embodiments, the HSV-2-specific meganuclease is configured to induce one DNA double strand break of an HSV-2 gene. In some embodiments, the HSV-2-specific meganuclease is configured to induce two DNA double strand breaks of an HSV-2 gene. In some embodiments, the HSV-2-specific meganuclease is configured to induce three DNA double strand breaks of an HSV-2 gene.

In some embodiments, each of the one or more DNA double strand breaks is effected by the same meganuclease sequence. In some embodiments, each of the two DNA double strand breaks is effected by the same meganuclease sequence. In some embodiments, each of the one or more DNA double strand breaks is effected by a different meganuclease sequence. In some embodiments, each of the two DNA double strand breaks is effected by a different meganuclease sequence.

Each of the DNA double strand breaks can result from any of one or more mechanism. For example, the HSV-2 DNA double strand breaks can remain cleaved from cleavage caused by the meganuclease. Alternatively, the HSV-2 DNA double strand breaks from meganuclease cleavage can be repaired, such as by non-homologous end joining, wherein repeated cleavage and repair of the HSV-2 DNA double strand breaks leads to DNA sequence errors, cleavage, or mutation of the HSV-2 gene. The HSV-2 DNA double strand breaks can lead to degradation and or loss of the HSV-2 genomes.

In some embodiments, the HSV-2-specific meganuclease targets one or more HSV-2 genes essential for replication. In some embodiments, the one or more HSV-2-specific meganuclease is configured to target one or more HSV-2 genes encoding proteins known to be essential for replication, for example in cells, in cell culture, or in vivo. In some embodiments, the one or more HSV-2-specific meganuclease is configured to target one or more HSV-2 genes encoding proteins not known to be essential for replication in cells, cell culture, or in vivo, or which are known to not be essential for replication in cells, in cell culture, or in vivo, but that may or may not be essential for human infection.

In some embodiments, the HSV-2-specific meganuclease targets one or more HSV-2 DNA sequences essential for replication. In some embodiments, the one or more HSV-2-specific meganuclease is configured to target one or more HSV-2 DNA sequences encoding proteins known to be essential for replication, for example in cells, in cell culture, or in vivo. In other embodiments, the one or more HSV-2-specific meganuclease is configured to target one or more HSV-2 DNA sequences not known to encode for proteins, or which are known to not encode for proteins. In still other embodiments, the one or more HSV-2-specific meganuclease is configured to target one or more HSV-2 DNA sequences encoding proteins known to be essential for replication in cells, in cell culture, or in vivo; one or more HSV-2 DNA sequences encoding proteins not known to be essential for replication in cells or cell culture, or are known to not be essential for replication in cells or cell culture, and that may or may not be essential for human infection; one or more HSV-2 DNA sequences not known to encode for proteins or are known to not encode for proteins; or a combination thereof.

The HSV-2 genes or DNA sequences essential for replication are estimated to comprise around 30-40% of the total HSV-2 genes, and are essential for productive replication in cultured cells. These essential genes can be broadly categorized into functions critical for viral entry (e.g., gB, gD, gH/L), viral DNA replication (e.g., DNA polymerase-UL30, helicase-primase complex-UL42, UL52), and virion assembly (e.g., capsid proteins-UL19, UL26, scaffolding proteins-US3). Notably, many essential genes are highly conserved across HSV-2 strains, making them attractive targets for meganuclease therapy.

In some embodiments, the one or more HSV-2-specific meganucleases is configured to target a duplicated portion of the HSV-2 LAT region with the sequence TATCCTTTTTTTCTAGGTGTTT (SEQ ID NO: 13), derived from OnuHSV2a. In some embodiments, the one or more HSV-2-specific meganucleases is configured to target the sequence TCTGCATCTATACCTCTTTCTG (SEQ ID NO: 14) derived from (OnuHSV2b). The OnuHSV2b gene target is in the HSV-2 gene UL48. In some embodiments, the one or more HSV-2-specific meganuclease comprises a meganuclease configured to target other duplicated portions of the HSV-2 genome. In some embodiments, the one or more HSV-2-specific meganuclease comprises a meganuclease configured to target a non-duplicated portion of the HSV-2 genome. In some embodiments, the one or more HSV-2-specific meganuclease comprises a meganuclease configured to target a combination of HSV-2 genome targets.

In some embodiments, the one or more viral vectors further includes one or more regulatory sequences. For example, the one or more viral vectors further includes the regulatory sequence as set forth in SEQ ID NO: 42.

CbH:
(SEQ ID NO: 42)
cgttacataacttacggtaaatggcccgcctggctgaccgcccaacgacc
cccgcccattgacgtcaatagtaacgccaatagggactttccattgacgt
caatgggtggagtatttacggtaaactgcccacttggcagtacatcaagt
gtatcatatgccaagtacgccccctattgacgtcaatgacggtaaatggc
ccgcctggcattgtgcccagtacatgaccttatgggactttcctacttgg
cagtacatctacgtattagtcatcgctattaccatggtcgaggtgagccc
cacgttctgcttcactctccccatctccccccetccccacccccaatttt
gtatttatttattttttaattattttgtgcagcgatgggggcgggggggg
ggggggcgcgcgccaggcggggggggcgaggggcggggggggcgaggcgg
agaggtgcggcggcagccaatcagagcggcgcgctccgaaagtttccttt
tatggcgaggcggcggcggcggcggccctataaaaagcgaagcgcgcggg
gggggagtcgctgcgcgctgccttcgccccgtgccccgctccgccgccgc
ctcgcgccgcccgccccggctctgactgaccgcgttactcccacaggtga
gcggggggacggcccttctcctccgggctgtaattagctgagcaagaggt
aagggtttaagggatggttggttggtggggtattaatgtttaattacctg
gagcacctgcctgaaatcactttttttcaggttgg.

The CbH promoter is identified by SEQ ID NO: 42, and is a strong constitutive promoter that drives high-level transgene expression in a variety of cell types, ensuring high-level expression of the meganuclease in neurons and other cell types.

In some embodiments, the composition further comprises a pharmaceutically acceptable carrier or excipient. “Pharmaceutically acceptable carrier,” “pharmaceutically acceptable excipient,” or “pharmaceutically acceptable carrier or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, pH adjusting agent, hydrogel, salt, inert solid, printed solid, semi-liquid, liquid, or emulsifier, which has been approved by the United States Food and Drug Administration as being acceptable for use in humans, mammals, or domestic animals. Each carrier must be “acceptable” in the sense of being compatible with the other ingredients of the formulation and not injurious to the subject.

In some embodiments, the composition disclosed herein and the pharmaceutically acceptable carrier or excipient comprise a “pharmaceutical composition,” wherein the pharmaceutical composition refers to a formulation of a compound of the disclosure and a medium generally accepted in the art for the delivery or administration of the compound to mammals, e.g., humans. Such a medium includes all pharmaceutically acceptable carriers or excipients as described herein. For example, the pharmaceutically acceptable carrier or excipient can be a carrier or excipient suitable for oral, nasal, rectal, patenteral, intracisternal, intravaginal, intraperitoneal, topical, subcutaneous, intramuscular, or buccal administration.

In some embodiments, the composition or pharmaceutical composition is configured for e.g., injection, oral administration, intravaginal, or other administration, to provide an effective amount of the composition or pharmaceutical composition to reduce or eliminate latent HSV-2 or HSV-2 reactivation in a cell or in a subject. In some embodiments, administration by injection is subcutaneous, intravenous, intraperitoneal, and/or intramuscular. In some embodiments, the injection includes injection into the cerebrospinal fluid, injection into ganglia, autologous cell transfer, and/or allogeneic cell transfer.

Injectable preparations include, for example, sterile injectable aqueous or oleaginous suspensions formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P. and isotonic sodium chloride solution. In addition, sterile, fixed oils can be employed as a solvent or suspending medium. For this purpose, any bland fixed oil can be employed including synthetic mono- or diglycerides. In addition, fatty acids such as oleic acid can be used in the preparation of injectables.

In another aspect, this disclosure provides a method of reducing or eliminating latent HSV-2 or HSV-2 reactivation in a cell, comprising administering the composition as described herein. Specifically, in some embodiments, the method for reducing or eliminating latent HSV-2 or HSV-2 reactivation in a cell comprises administering to an HSV-2-infected cell a plurality of one or more viral vectors, wherein each of the one or more viral vectors includes a sequence encoding an HSV-2-specific meganuclease, as described herein.

In some embodiments, the method of reducing or eliminating latent HSV-2 or HSV-2 reactivation in a cell comprises administering the composition to an HSV-2 infected cell in an amount effective to reduce or eliminate latent HSV-2 or HSV-2 reactivation in the cell.

In some embodiments, the cell is a mammalian cell. In some embodiments, the cell is a human cell. In some embodiments, the composition is administered to an HSV-2-infected cell in a mammalian subject. In some embodiments, the mammalian subject is human.

In some embodiments, the HSV-2 infected cell is a neuron. In some embodiments, the cell is a sensory ganglia cell. In some embodiments, the cell is an autonomic ganglia cell. In some embodiments, the cell is a trigeminal ganglia cell. In some embodiments, the cell is a dorsal root ganglia cell. In some embodiments, the cell is a superior cervical ganglia cell. In some embodiments, the cell is a major pelvic ganglia cell. In some embodiments, the cell is a neuron within ganglia, for example, the ganglia disclosed herein. In some embodiments, the HSV-2-infected cell is a combination of more than one different type of HSV-2-infected cell. In some embodiments, the HSV-2-infected cell comprises trigeminal ganglia, dorsal root ganglia, superior cervical ganglia, major pelvic ganglia, sensory ganglia, autonomic ganglia, other HSV-2-infected cells, or a combination thereof. In some embodiments, the HSV-2-infected cell comprises sensory ganglia, autonomic ganglia, or a combination thereof. In some embodiments, the HSV-2-infected cell comprises trigeminal ganglia, dorsal root ganglia, superior cervical ganglia, major pelvic ganglia, or a combination thereof.

In some embodiments, the HSV-2-infected cell is a superior cervical ganglia cell, a trigeminal ganglia cell, a dorsal root ganglia cell, a major pelvic ganglia cell, or a combination thereof.

In some embodiments, the one or more viral vectors can be delivered to the cell according to methods generally well known to one of ordinary skill in the art, and are appropriate for the particular viral vector and cell type.

In another embodiment, this disclosure provides a method of reducing or eliminating latent HSV-2 or HSV-2 reactivation in a subject, comprising administering the composition as described herein. Specifically, in some embodiments, the method for reducing or eliminating latent HSV-2 or HSV-2 reactivation in a subject comprises administering to a subject infected with HSV-2 a plurality of one or more viral vectors, wherein each of the one or more viral vectors includes a sequence encoding an HSV-2-specific meganuclease, as described herein.

In some embodiments, the plurality of one or more viral vectors comprising a sequence encoding an HSV-2-specific meganuclease is administered in an amount effective to reduce or eliminate latent HSV-2 or HSV-2 reactivation in the subject.

In some embodiments, the method of reducing or eliminating latent HSV-2 or HSV-2 reactivation in a subject comprises administering to the subject a pharmaceutical composition, wherein the pharmaceutical composition comprises a plurality of one or more viral vectors which comprise a sequence encoding an HSV-2-specific meganuclease, and a pharmaceutically acceptable carrier or excipient as described herein.

In some embodiments, the subject is a mammal. In some embodiments, the subject is a human.

As used herein, “administering” refers to administering the one or more viral vectors comprising a sequence encoding an HSV-2-specific meganuclease into a subject by a method or route which is effective in reducing or eliminating HSV-2 in the subject, and results in complete or partial inoculation of the subject, or of the subject at the desired site. Complete inoculation means no amount of HSV-2 is detectable by the methods known or used by a person having skill in the art. Partial inoculation means the amount of HSV-2 in the subject is decreased relative to the amount of HSV-2 present in the subject prior to the administering. For example, the amount of HSV-2 in the subject is decreased by about 10% to about 99%, about 15% to about 99%, about 20% to about 99%, about 30% to about 99%, about 40% to about 99%, about 50% to about 99%, about 60% to about 99%, about 70% to about 99%, about 80% to about 99%, about 90% to about 99%, about 10% to about 98%, or about 20% to about 95%.

In some embodiments, an “effective” amount of the viral vector is delivered, or administered, to modify the HSV-2 genome. As used herein, the term “effective” refers to any amount that induces the desired response while not inducing significant toxicity in the subject. For example, an “effective” amount is an amount which modifies the HSV-2 genome in about 10% to about 100%, about 15% to about 100%, about 20% to about 100%, about 30% to about 100%, about 40% to about 100%, about 50% to about 100%, about 60% to about 100%, about 70% to about 100%, about 80% to about 100%, about 90% to about 100%, about 10% to about 98%, about 20% to about 95% of cells in which the composition is in contact. An “effective” amount is also an amount which modifies the HSV-2 genome in cells such that no amount of HSV-2 is detectable by HSV-2 detection methods either known or used in the art.

An “effective” amount is also an amount which results in a decreased number of HSV-2 infected cells relative to the number of HSV-2 infected cells present prior to administering the viral vector. For example, an “effective” amount is an amount which decreases the number of HSV-2-infected cells by about 10% to about 100%, about 15% to about 100%, about 20% to about 100%, about 30% to about 100%, about 40% to about 100%, about 50% to about 100%, about 60% to about 100%, about 70% to about 100%, about 80% to about 100%, about 90% to about 100%, about 10% to about 98%, or about 20% to about 95%.

The terms “decrease” or “reduce” are all used herein generally to mean a decrease by a statistically significant amount. For example, “decrease” or “reduce” each mean a decrease by at least 10% as compared to a reference level, a decrease by at least about 20%, at least about 30%, at least about 40%, at least about 50%, at least about 60%, at least about 70%, at least about 80%, at least about 90%, about 100%, or up to and including a 100% decrease (e.g. absent level, or having a non-detectable level, as compared to a reference level), or any decrease between 10-99% as compared to a reference level. “Eliminate” means complete removal or wherein no amount is detectable.

The use of the term “or” in the claims is used to mean “and/or” unless explicitly indicated to refer to alternatives only, or the alternatives are mutually exclusive.

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense, and to indicate “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application.

The term “subject” herein encompasses, without limitation, a mammal having HSV-2 infection, latent HSV-2 infection, or latent HSV-2 reactivation; a mammal being assessed for reducing or eliminating HSV-2 infection, latent HSV-2 infection, or latent HSV-2 reactivation; a mammal suspected of having HSV-2 infection, latent HSV-2 infection, or latent HSV-2 reactivation; or a mammal at risk of having HSV-2 infection, latent HSV-2 infection, or latent HSV-2 reactivation.

In some embodiments, the subject is one who is at a risk of developing an HSV-2 infection, is diagnosed with HSV-2, is seeking treatment for HSV-2, is currently being treated for HSV-2, is currently being treated for HSV-2 and is seeking monitoring or modification of the existing therapeutic treatment, has previously been treated for HSV-2, or has been infected with HSV-2 regardless of having previously received any treatment for HSV-2.

As used herein, the term “HSV-2 infection” refers to the undesired proliferation or presence of HSV-2 in a host organism or cell, or a subject. Infection can be caused by actively replicating lytic HSV-2, and can be referred to as lytic infection. Infection can be caused by quiescent or latent HSV-2, and can be referred to as latent HSV-2 infection. A latent infection can reactivate to become a lytic infection or recurrent HSV-2 infection, and can result in recurrence of active symptomatic HSV-2-related disease.

As used herein, the term “protein” refers to a series or polymer of amino acid residues connected to each other by peptide bonds between the alpha-amino and carboxy groups of adjacent amino acid residues. The term “protein” can also refer to modified amino acids (e.g., phosphorylated, methylated, glycosylated, and the like), and amino acid analogs, regardless of size or function. The term “protein” can also be used to refer to a gene product and/or fragments thereof.

As used herein, the term “pharmaceutically acceptable” refers to those compounds, materials, compositions, and/or dosage forms which are within the scope of sound medical judgment and suitable for use in contact with the tissues of human beings and animals without excessive toxicity, irritation, allergic response, or other problem or complication, and commensurate with a reasonable benefit/risk ratio.

### EXAMPLES

The following examples are set forth to provide those of ordinary skill in the art with a complete disclosure and description of how to make and use the present invention. The following examples are not intended to limit the scope of the invention, nor are they intended to represent the entire scope of experiments performed.

### Example 1

### Engineering of Meganucleases

The thermostabilized I-Onul meganuclease has been successfully engineered to target the OnuHSV2a DNA sequence TATCCTTTTTTTCTAGGTGTTT (SEQ ID No: 13) and the OnuHSV2b DNA sequence TCTGCATCTATACCTCTTTCTG (SEQ ID No: 14). The OnuHSV2a target is a duplicated target site, meaning that a single meganuclease targeting this sequence would create two separate cuts in the viral genome. The OnuHSV2b target site exists only once in the HSV-2 genome. These custom-specificity meganucleases were engineered through iterative rounds of structure-based directed evolution combined with expression on the surface of yeast and selection for the desired DNA-cleaving activity using fluorescence activated cell sorting (FACS) and a flow cytometric DNA cleavage assay.

### Target Site Selection

The HSV-2 viral genome was searched for 22 basepair target sequences with up to ten mismatches away from the wildtype DNA recognition sequence for the I-OnuI meganuclease (TTTCCACTTATTCAACCTTTTA SEQ ID NO: 15). Targets were only considered if any mismatches within the central four basepairs of the recognition sequence fell within the list of known tolerated central-four sequences for I-Onul. Target sequences were considered good if they were located within essential genes for HSV-2 and were prioritized if the target sequence was also a duplicated target site, meaning that a single meganuclease would cut the HSV-2 genome at two separate sites. For each HSV-2 target sequence under consideration, sequence database searching tools were used to identify any closely-related DNA sequences (3 mismatches or less) in both the human and mouse genomes.

### Library Design Strategy

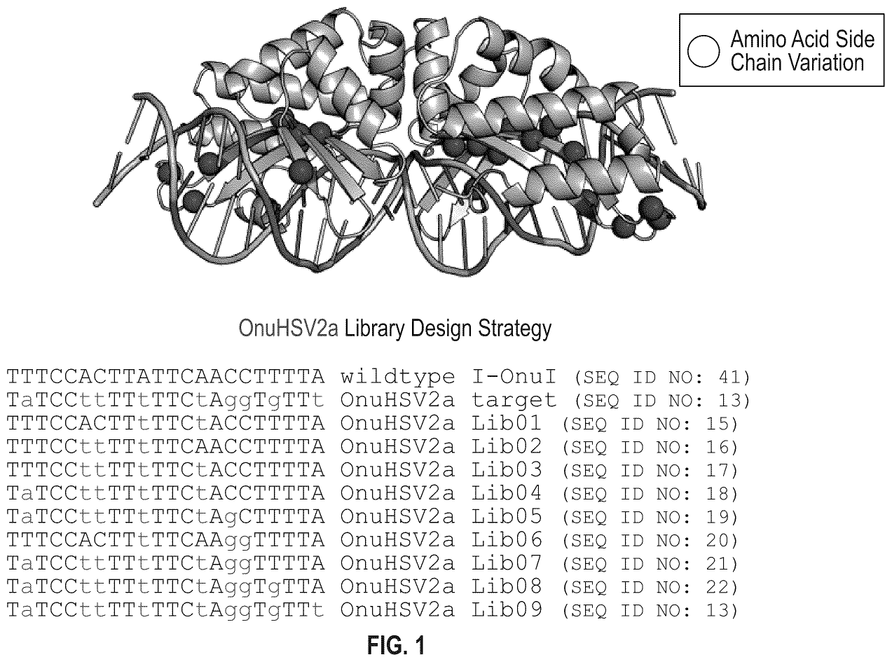

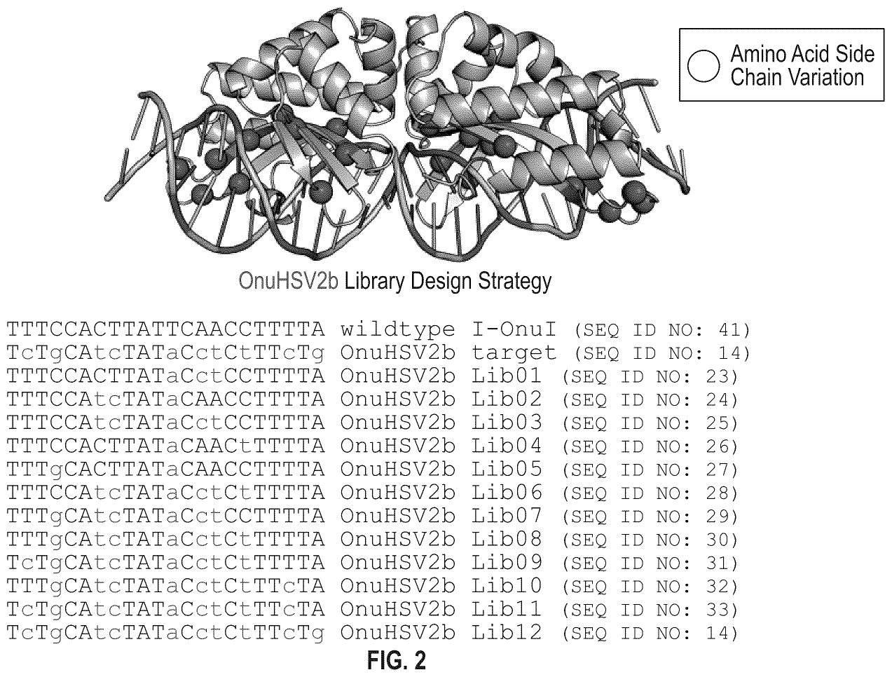

The desired 22 bp HSV-2 target sequence was aligned to the wildtype DNA recognition sequence of the I-Onul meganuclease. The locations of mismatches were highlighted, and a series of engineering libraries was designed to make small (across no larger than a 3 bp window at a time), stepwise changes to the meganuclease, starting from the center of the target site (nearest the active site of the enzyme) and moving progressively outward. Small changes were designed on each half of the target site separately and then combined across the center of the target before progressing outward. See FIG. 1 for the list of libraries designed for engineering towards the HSV-2 target “OnuHSV2a” (SEQ ID NO: 13) and FIG. 2 for the library design strategy for the HSV-2 DNA target “OnuHSV2b” (SEQ ID NO: 14). The OnuHSV2a DNA target site contains 9 mismatches from the wildtype I-Onul recognition sequence, with one of those mismatches being a tolerated alternative central four sequence. The OnuHSV2b DNA target site contains 10 mismatches, with one of those mismatches being a tolerated alternative central four sequence.

The crystal structure of the DNA-bound thermostabilized I-Onul meganuclease (PDB ID 6UVW) was used to identify the individual amino acid sidechains contacting the bases of the DNA target site where changes were required. Degenerate codons were incorporated into the meganuclease coding sequence using assembly PCR, and the resulting collection of sequences were recombined into the pETCON yeast surface display vector to create libraries of the meganuclease with variation at specific positions in the amino acid sequence.

Libraries were generated and screened. Briefly, library insert DNA with degenerate codons at strategically-placed positions was combined with open pETCON yeast surface display vector and transformed into EBY100 yeast using the lithium acetate transformation method. Expression of the library of meganucleases on the yeast surface was induced through growth in selective media containing galactose.

Libraries were screened for the desired DNA cleavage activity using a tethered flow cytometric activity assay. A double-stranded DNA substrate containing the desired DNA target sequence was generated by PCR amplification using one biotinylated primer and a second primer conjugated to the Alexa647 (A647) fluorophore. This created a DNA target substrate with biotin at one end and a fluorescent A647 tag at the opposite end. The PCR product was incubated with Exonuclease I to digest away any unincorporated primers and then purified over a small size exclusion column made from Sephadex G-100 resin.

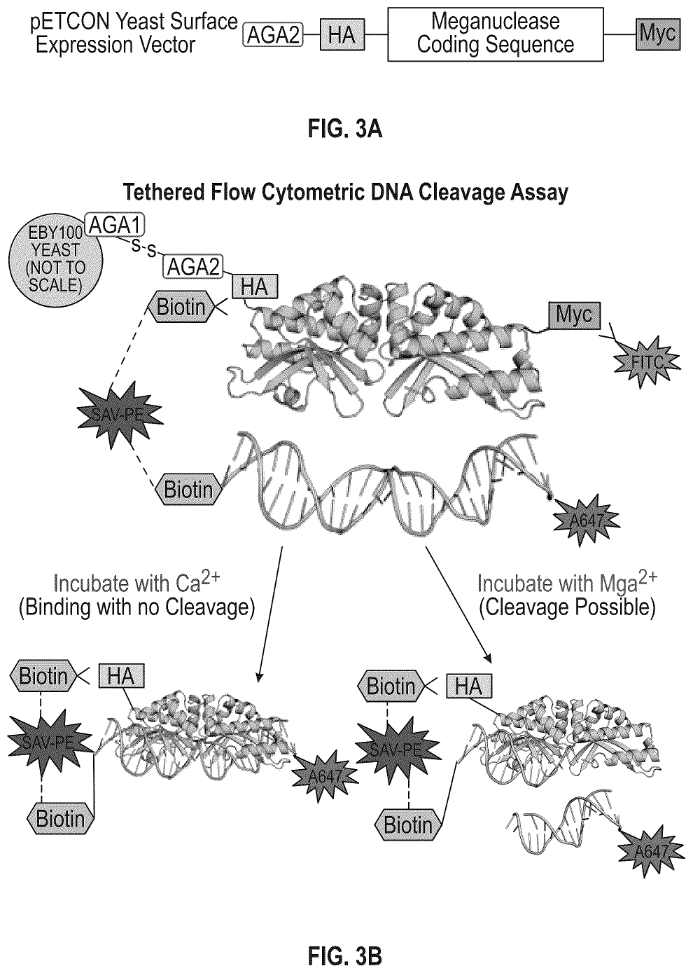

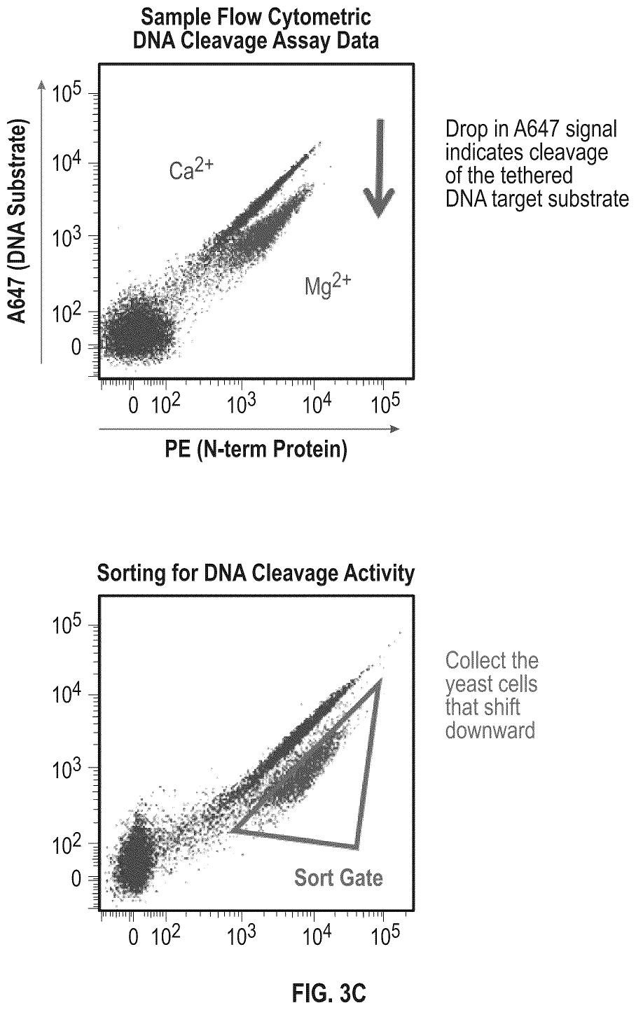

The pETCON yeast surface display vector expresses the meganuclease coding sequence with an N-terminal hemagglutinin (HA) epitope tag and a C-terminal Myc epitope tag (FIG. 3A). In a primary stain, the HA tag was stained with an anti-HA-biotin antibody and the Myc tag with a fluorescent anti-Myc-FITC antibody (FIG. 3B). Signal from the FITC fluorophore can be measured to detect stable, full-length protein expression on the yeast surface. The biotin-labeled DNA target substrate was pre-conjugated at 40 nM concentration with 5 nM streptavidin-phycoerythrin (SAV-PE). The pre-conjugated DNA substrate was then incubated with primary-stained yeast cells to create a physical tether at the N-terminus of the meganuclease between the anti-HA-biotin antibody and the biotinylated DNA substrate conjugated to the SAV-PE (FIG. 3B).

The yeast library was incubated with a cleavage buffer containing calcium to allow the surface-expressed meganucleases to bind the tethered substrate (but not cleave it), or a buffer containing magnesium to allow for cleavage to occur. If a surface-expressed meganuclease is able to cleave its tethered DNA substrate, the fluorescent A647 tag on the DNA is released and washed away (FIG. 3C). The physical tethering of the DNA target substrate in the presence of calcium creates a sharp diagonal line in a flow cytometric plot of A647 signal vs. PE signal (FIG. 3C). Cleavage of the tethered substrate in the presence of magnesium leads to a drop in the A647 signal. This loss of A647 signal creates a distinct population of yeast cells dropping down from the diagonal, and a gate can be drawn on a cell sorter to capture the yeast library variants which have successfully cleaved their tethered DNA target substrates (FIG. 3C).

For each library, the tethered flow cytometric DNA cleavage assay was used in two subsequent rounds of sorting to collect meganuclease variants which were active against the desired DNA target substrate. The first sort was performed on the full yeast library with a digest at 37° C. at pH 7.2 for 45 minutes. The sorted cells were grown up in rich media and then re-induced for surface expression in galactose-containing selective media. The second sort was then performed on the first-sort enriched culture at more stringent digest conditions of 37° C. at a lower pH of 7.0 for a shortened time of 30 minutes. The sort gate was drawn further down to collect only the yeast with the largest drop in A647 signal.

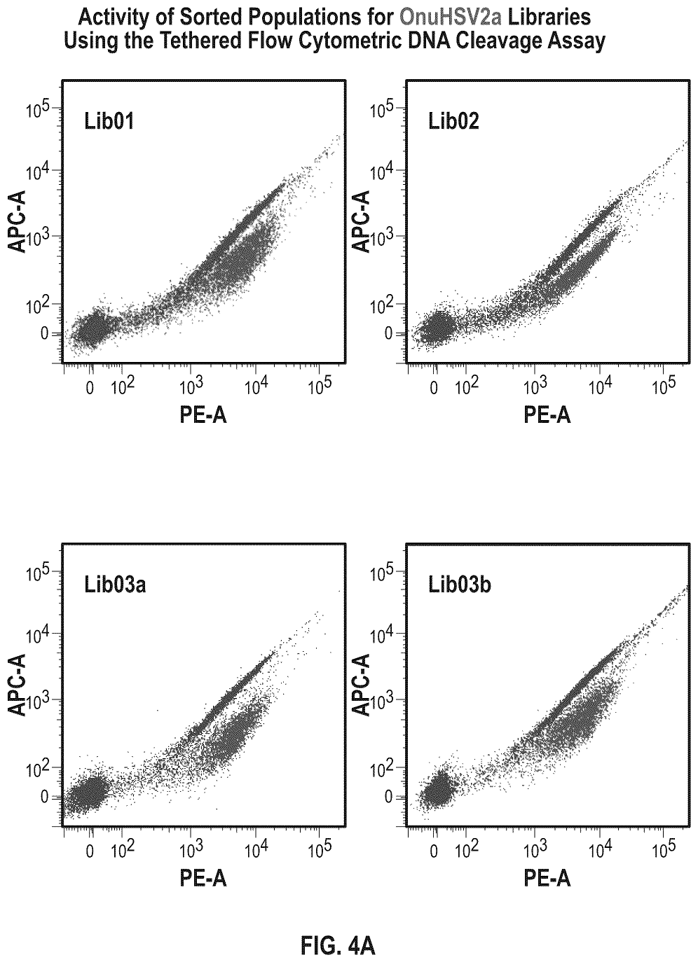

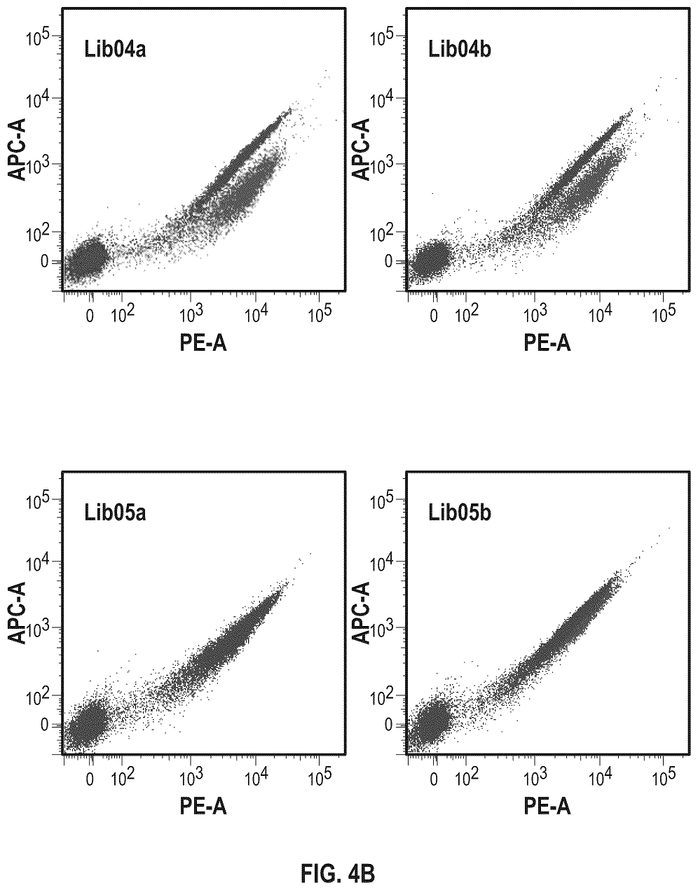

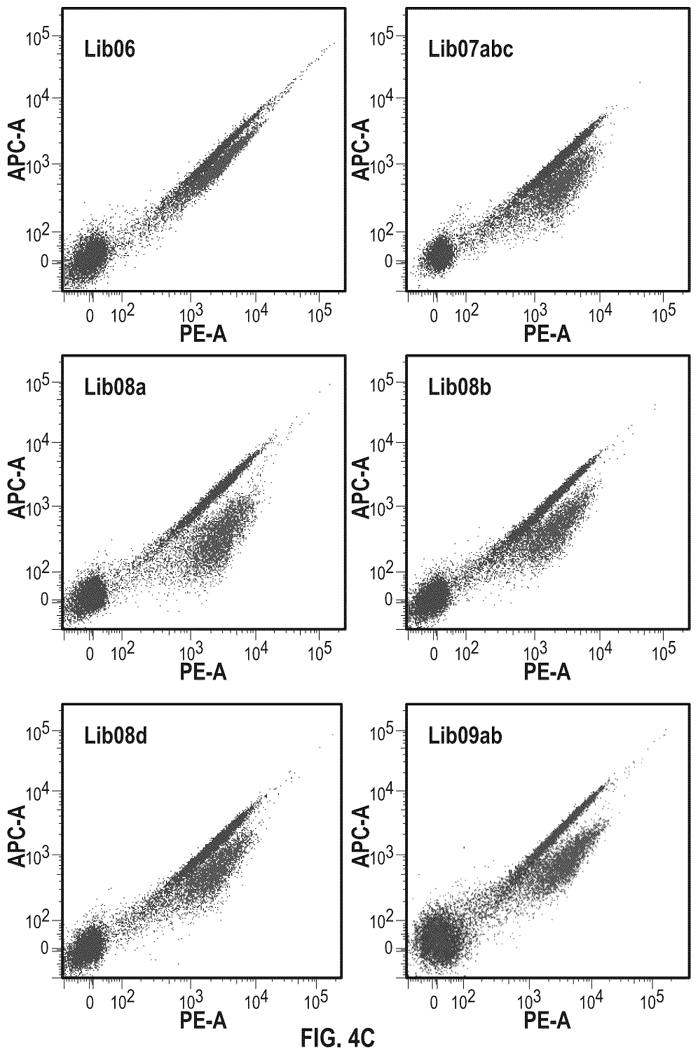

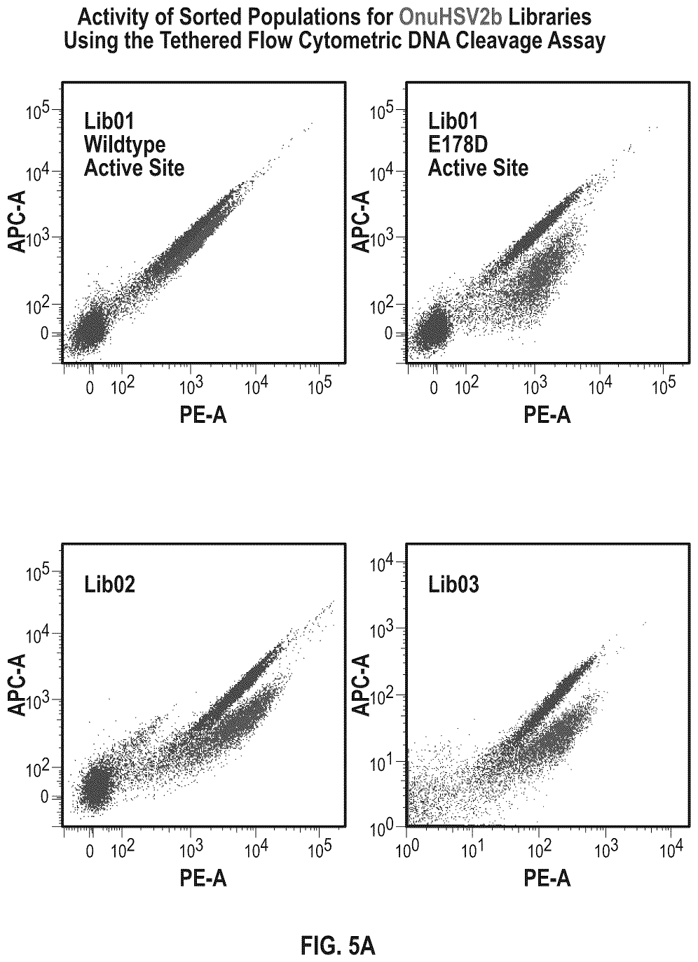

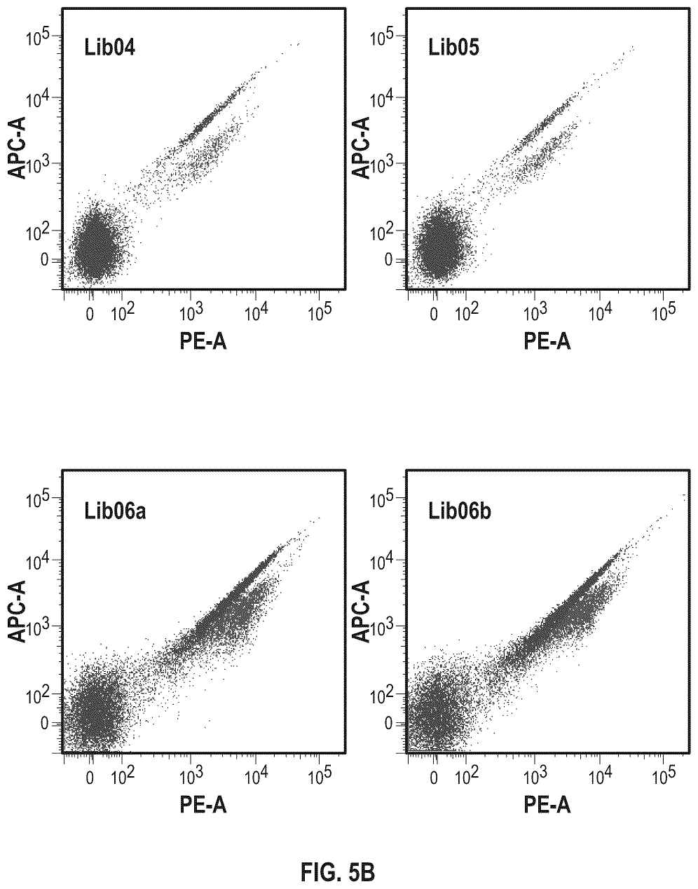

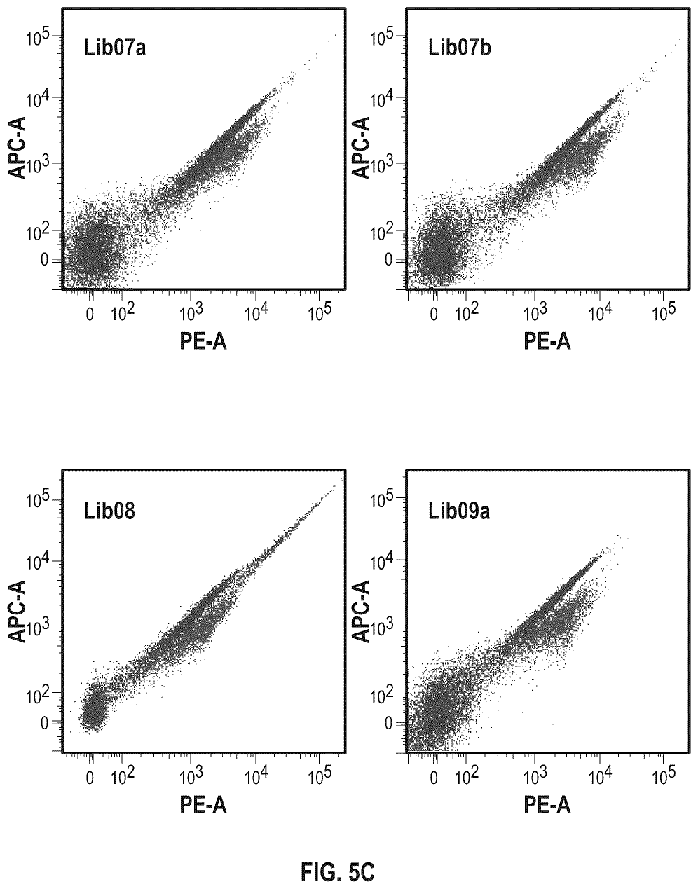

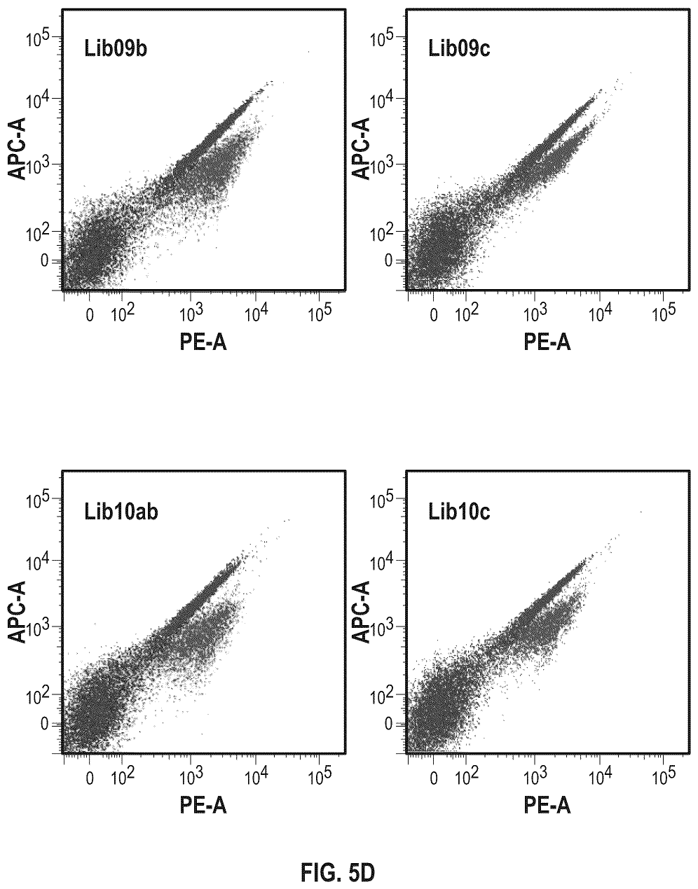

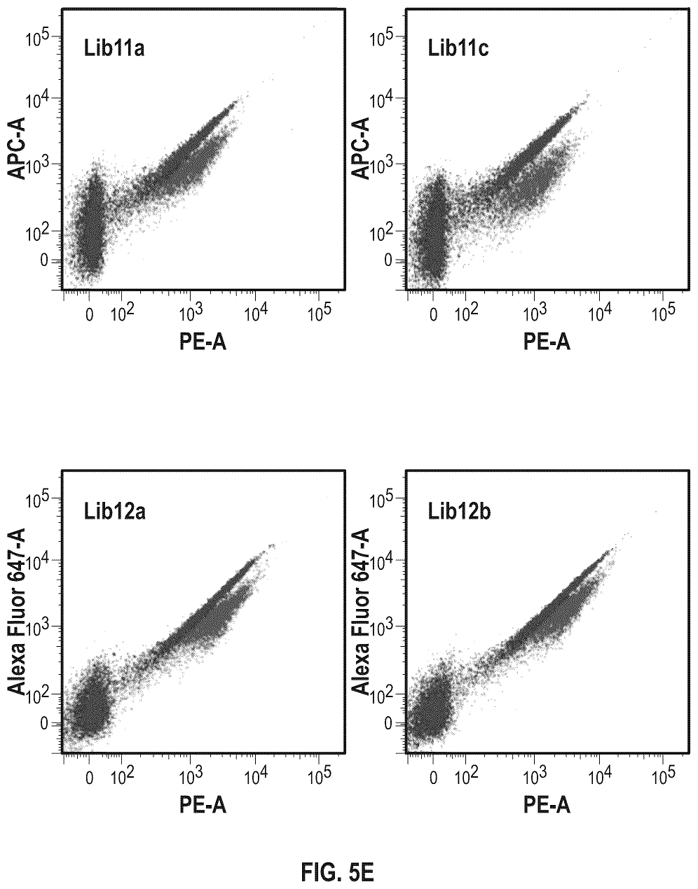

After two rounds of sorting for each library, the tethered flow cytometric DNA cleavage assay was performed on the final sorted population to verify enrichment of meganuclease variants with cleavage activity against the desired DNA target. See FIG. 4 for the results of the activity checks of the sorted populations after each of the 9 libraries required for the OnuHSV2a target. See FIG. 5 for the results of the activity checks of the sorted populations after each of the 12 libraries required for the OnuHSV2b target.

A yeast mini-prep was performed on the final sort population for each library, and the extracted pETCON plasmid DNA was transformed into chemically competent bacteria. This step assumes that each resulting bacterial colony contains the meganuclease-encoding pETCON plasmid from a single sorted yeast cell. Individual bacterial colonies were used to inoculate small storage cultures and sequenced via colony PCR. The resulting meganuclease sequences were aligned to look for unique variants.

Once unique variant meganuclease sequences were identified, the bacterial colony storage cultures were used to perform bacterial plasmid minipreps for each variant meganuclease. The plasmid preps were transformed individually back into yeast using the lithium acetate method, and cultures for each unique meganuclease variant were grown up and induced for expression on the yeast surface.

### Example 2

### Meganuclease DNA Cleavage

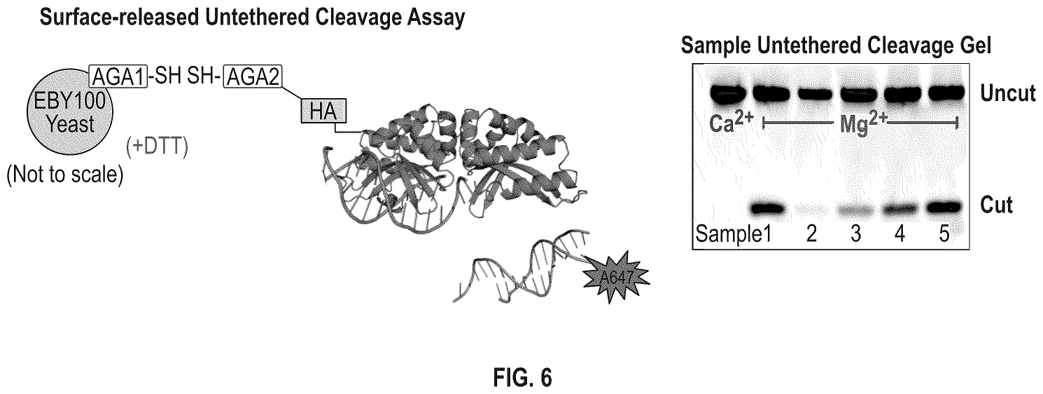

Two separate activity assays were used to perform a side-by-side comparison of meganuclease DNA cleavage activity against the desired DNA target. The tethered flow cytometric cleavage assay (previously described) was performed alongside a surface-released DNA cleavage activity assay (FIG. 6). In this non-tethered cleavage assay, the same surface-expressed yeast cultures are used, but the addition of dithiothreitol (DTT) to the reaction releases the meganuclease from the surface of yeast and allows it to float freely in solution. The same biotin/A647-labeled DNA target substrates are added to the reaction, but there is no physical tethering of the substrate to the meganuclease. Therefore, the meganuclease must be able to both bind and cleave the DNA target substrate for cleavage activity to be observed. The non-tethered DNA cleavage activity assay can be considered a more realistic representation of the meganuclease's ability to interact with its desired DNA target site. The physical tethering of the substrate in the flow cytometric cleavage assay can produce an artificially high cleavage activity for a meganuclease which would otherwise be binding-compromised. These two DNA cleavage assays are performed together on the same surface-expressed yeast cultures when the most complete assessment of binding and cleavage activity is required.

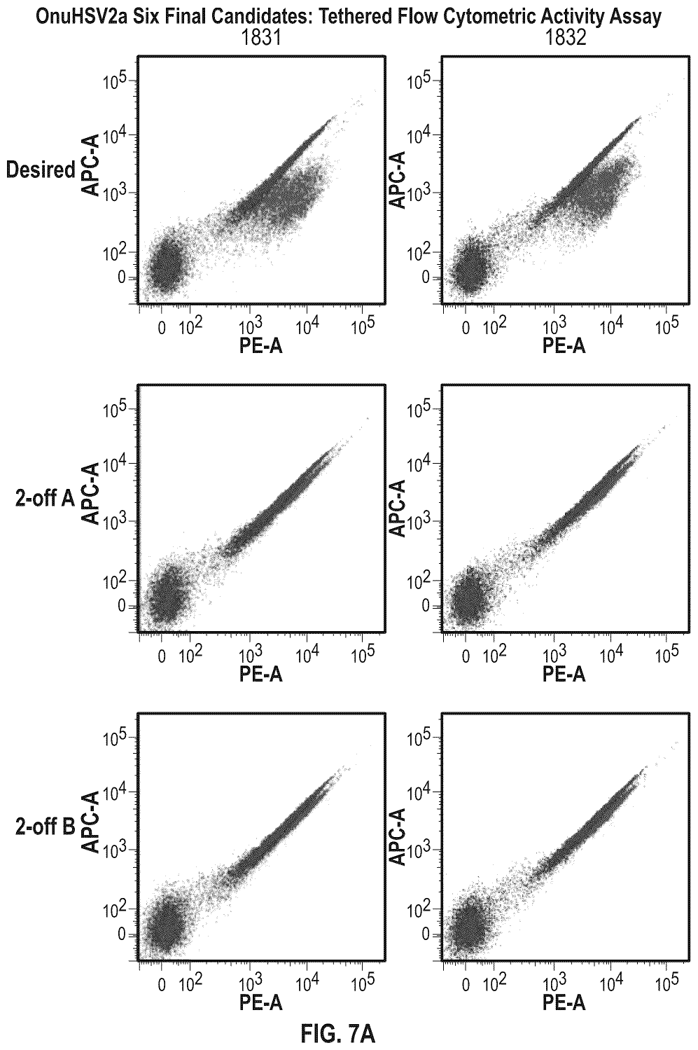

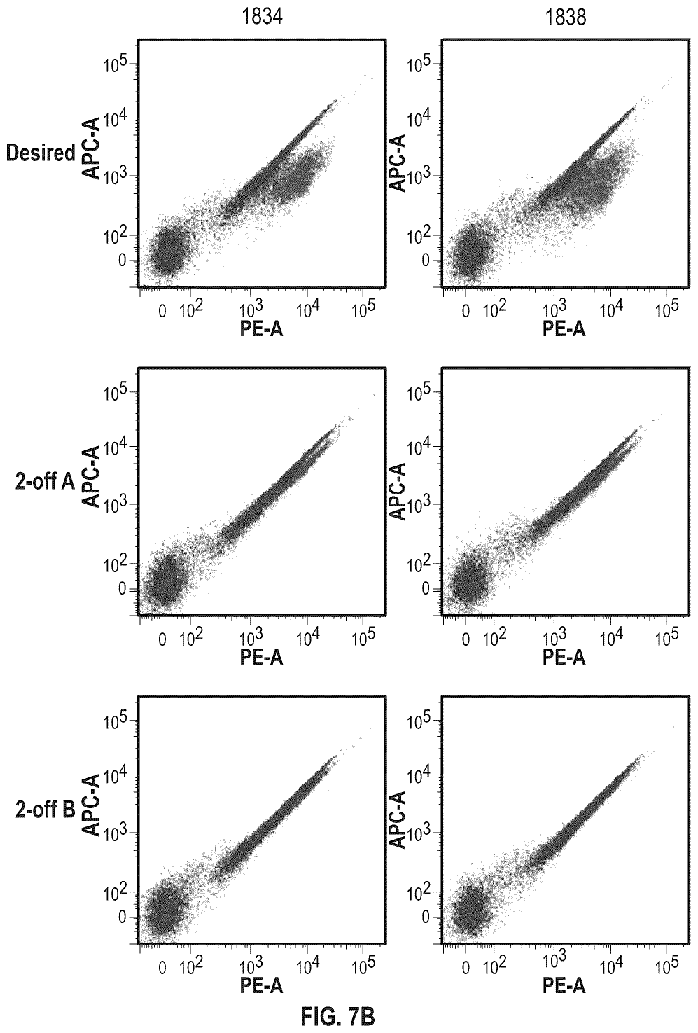

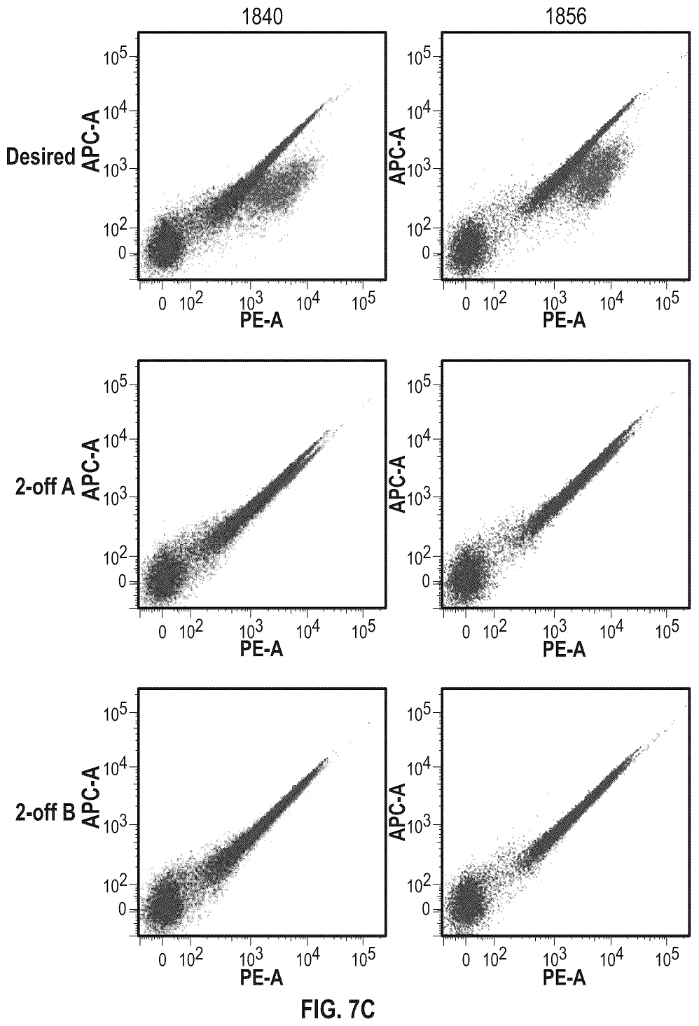

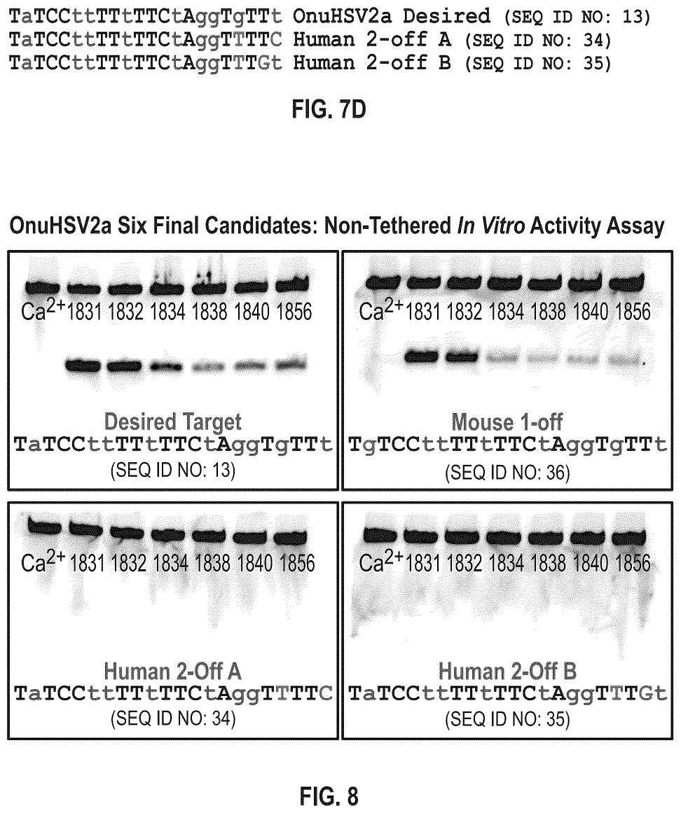

From the final library for the OnuHSV2a target site, 48 colonies were sequenced and 44 unique meganuclease variants were identified. These 44 unique variants were tested side-by-side for activity in both the tethered flow cytometric cleavage assay (FIG. 7) and the non-tethered DNA cleavage assay (FIG. 8) vs. the desired target and vs. three different off-target sequences. Off-target searching had identified two potentially troublesome “2-off” targets in the human genome and one “1-off” target in the mouse genome. Off-targets are concerning and need to be screened when the mismatches are located toward the outermost ends of the DNA recognition sequences. Meganucleases have a natural tendency to tolerate more sequence variation at these outer positions.

Of the 44 unique OnuHSV2a variants isolated, only 6 showed minimal to no activity vs. the two off-target sequences in the human genome (FIGS. 7 and 8) while maintaining activity against the desired HSV-2 target sequence. All of these 6 variants showed activity vs. the mouse 1-off sequence. These six engineered meganuclease variants were designated the final candidates for the OnuHSV2a target site and moved on to testing in mammalian cell lines.

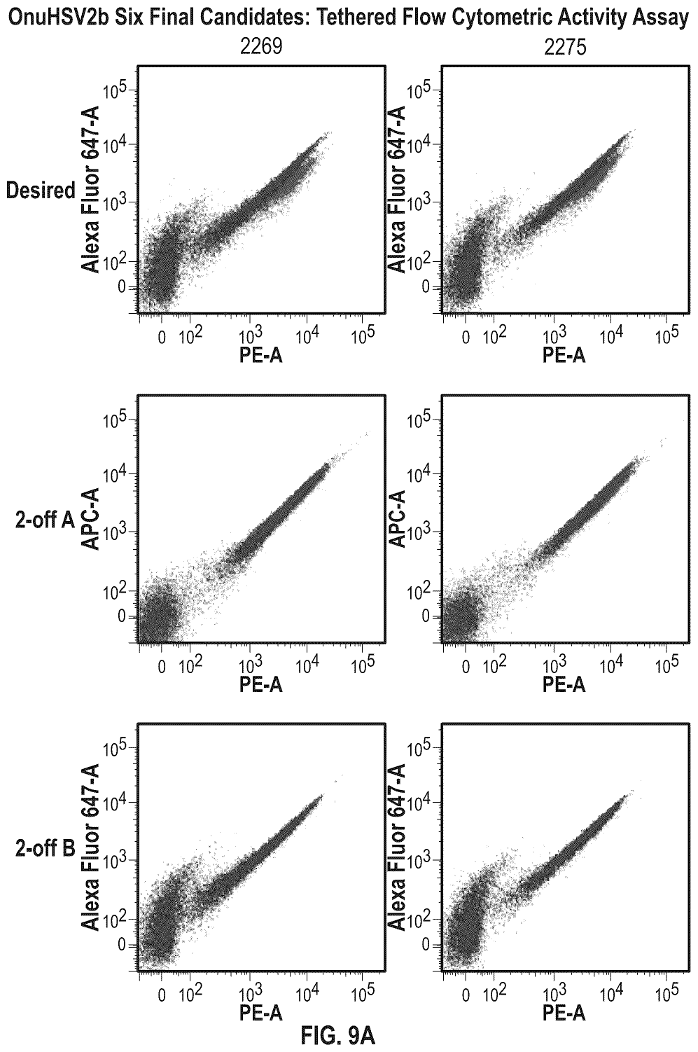

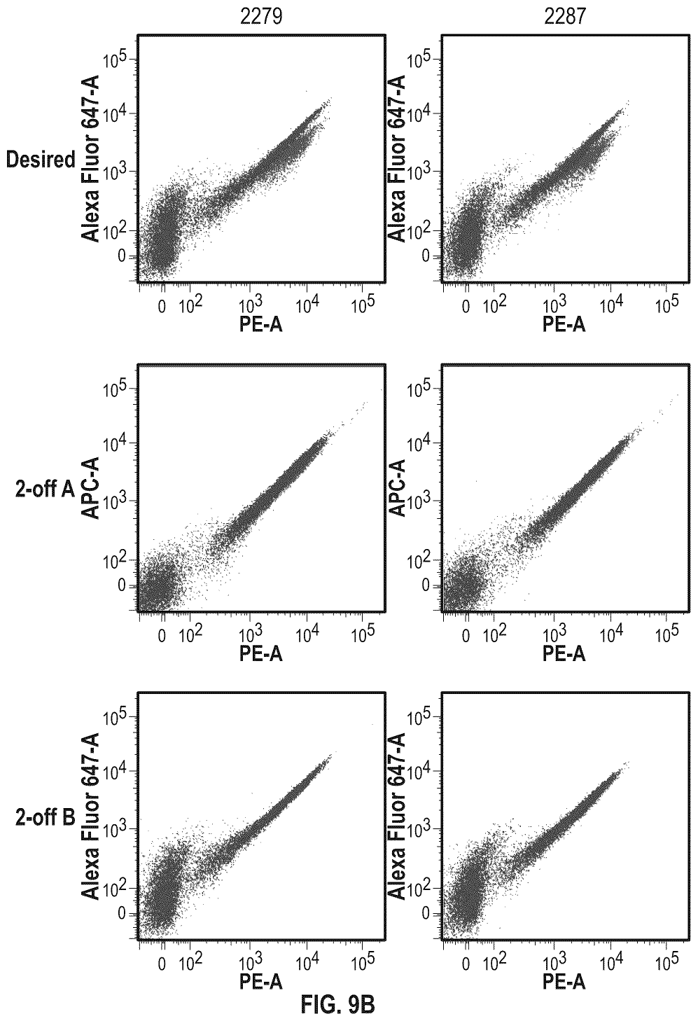

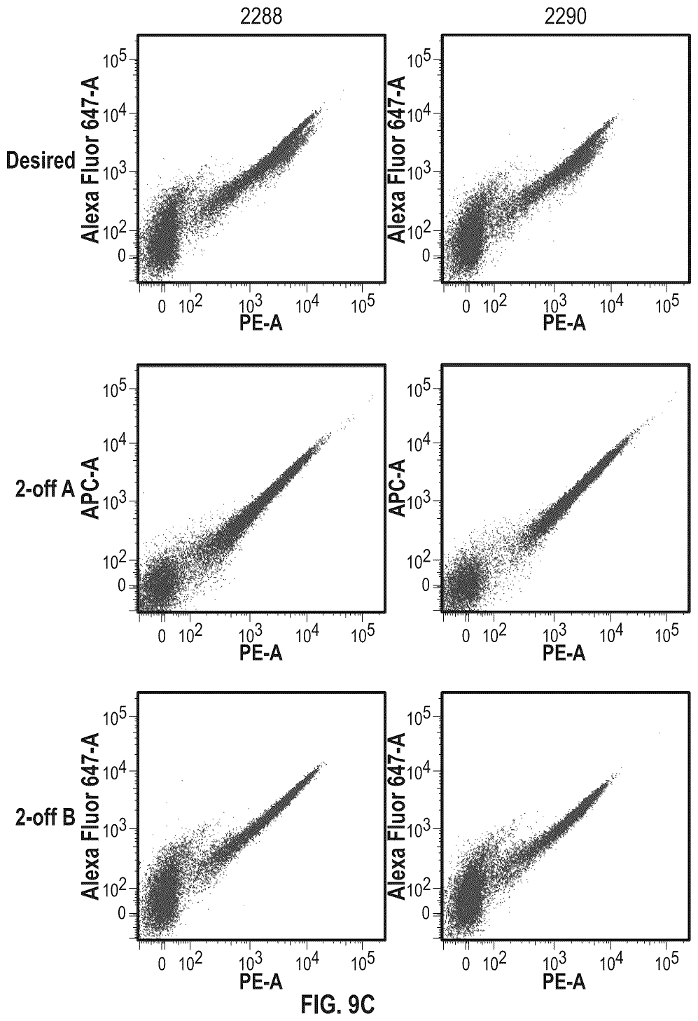

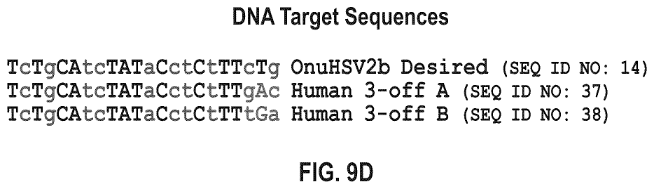

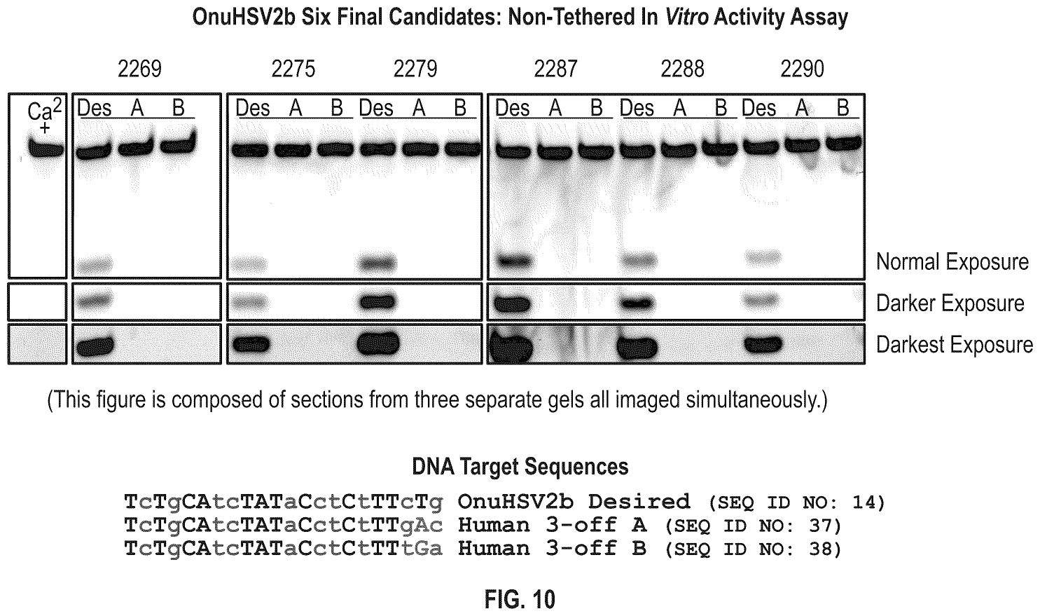

From the final library for the OnuHSV2b target site, 104 colonies were sequenced, and a total of 83 unique meganuclease sequences were identified. These 83 unique variants were tested side-by-side for activity vs. the desired target and vs. two different off-target sequences identified in the human genome in both the tethered flow cytometric cleavage assay (FIG. 9) and the non-tethered DNA cleavage assay (FIG. 10). Of the meganucleases tested, six were identified to have minimal to no activity vs. the two off-targets while maintaining activity against the desired HSV-2 target sequence. These six engineered meganuclease variants were designated the final candidates for the OnuHSV2b target site and moved on to testing in mammalian cell lines.

### Example 3

### Gene Editing Reduces Ganglionic HSV

This Example demonstrates that AAV-delivered meganucleases, but not CRISPR/Cas9, mediate highly efficient gene editing of HSV-2, eliminating over 90% of latent virus from superior cervical ganglia, and reducing HSV-2 shedding from infected animals. Single-cell RNA sequencing demonstrates that both HSV-2 and individual AAV serotypes are non-randomly distributed among neuronal subsets in ganglia, implying that improved delivery to all neuronal subsets may lead to even more complete elimination of HSV-2.

To improve endonuclease-directed gene editing of latent HSV genomes to levels needed for therapeutic benefit, the self-complementary scAAV vectors, simultaneous targeting of multiple sites within the HSV-2 genome, substitution of CRISPR/Cas9 for meganucleases, and the relative distribution of HSV-2 compared with AAV vectors at the single-cell level suggest meganuclease-mediated gene editing represents a plausible pathway toward cure of latent HSV-2. Application of meganuclease-mediated gene editing to HSV-2 requires the development of HSV-2 specific meganucleases as described herein.

### Methods

### Cells and Herpesviruses

HEK293 and Vero cell lines were propagated in Dubelcco's modified Eagle medium supplemented with 10% fetal bovine serum. HSV-2 strain F or syn17+ were used for the experiments and were propagated and titered on Vero cells.

### AAV Production and Titering

The following AAV vector plasmids were used to generate the AAV stocks in this study: pscAAV-CBh-m5, pscAAV-CBh-m8, pscAAV-CBh-m4, pssAAV-smCBA-m5-T2A-Trex2-2A-mCherry, pssAAV-sCMV-SaCas9-U6-sgRNA, pssAAV-CMV-SaCas9-U6-sgRNA, pssAAV-nEF-SaCas9-U6-sgRNA, pscAAV-CBh-NLS-mScarlet, pscAAV-CBh-NLS-mEGFP, pscAAV-CBh-NLS-DsRed-Express2, and pscAAV-CBh-NLS-mTagBFP2. AAV stocks of all serotypes were generated by transiently transfecting 293 cells using PEI at a ratio of 4:1 (μl PEI: μg DNA). Briefly, 1.6×10⁷ HEK293 cells were transfected with 28 μg DNA comprised of the DNA for a scAAV or ssAAV vector plasmid, a plasmid that expresses the AAV rep and capsid proteins, and a helper plasmid that expresses adenovirus helper proteins (pHelper) at the ratio of 5:1:3, respectively. At 24 hours post-transfection, media was changed to serum-free DMEM and after 72 hours cells were collected and re-suspended in AAV lysis buffer (50 mM Tris, 150 mM NaCl, pH 8.5) before freeze-thawing 4 times. AAV stocks were purified by iodixanol gradient separation. Recombinant adeno-associated virus purification using novel methods improves infectious titer and yield. The samples were concentrated into PBS using an Amicon Ultra-15 column and stored at −80° C. All AAV vector stocks were quantified by qPCR using primers/probe against the AAV ITR, with linearized plasmid DNA as a standard. AAV stocks were treated with DNase I and Proteinase K prior to quantification.

### Establishment of Neuronal Cultures

Neuronal cultures were established from TG harvested from mice at day 7 post-infection with 2×10⁵ PFU HSV (F) onto scarified cornea. Briefly, neuronal cultures were established after enzymatic digest with collagenase and dispase and purification of the resulting cell homogenates using a percoll gradient (12.5% and 28%). Neurons were counted and plated on poly-D-lysine- and laminin-coated 12 mm round slides at a density of 4,000 neurons per well. Neurons were cultured without removing the non-neuronal cells that provide important growth support, and therefore these cultures contained a mixed population of neurons, satellite glial cells and other cell types. Cultures were maintained with complete neuronal medium, consisting of Neurobasal A medium supplemented with 2% B27 supplement, 1% PenStrep, L-glutamine (500 μM), and nerve growth factor (NGF; 50 ng/ml). Medium was replaced every 2-3 days with fresh medium. Acyclovir (100 nM) was added to the culture medium for the first 5 days.

### HSV Infection and AAV Inoculation of Mice

Mice were housed in accordance with the institutional and NIH guidelines on the care and use of animals in research. 6-8 week old female Swiss Webster mice were used for all studies. For ocular HSV infection mice anesthetized by intraperitoneal injection of ketamine (100 mg per kg) and xylazine (12 mg per kg) were infected with 2×10⁵ PFU of HSV2 (F) or syn17+ following corneal scarification of the right eye using a 28-gauge needle. For AAV inoculation, mice anesthetized with ketamine/xylazine were unilaterally administered the indicated AAV vector dose by either intradermal whisker pad (WP) injection, retro-orbital (RO) or tail-vein (TV) injection. The right (ipsilateral) TG and both SCGs were collected at the indicated time. Presence of AAV in ganglia of treated mice was confirmed by ddPCR.

### Tissue Explant Reactivation

HSV was reactivated by incubating collected TG and SCG in 10% FBS-DMEM culture medium for 24 h, followed by total genomic DNA extraction as described below. After tissue reactivation, a statistically significant increase of 2 to 3-fold in HSV genomes was detected in reactivated tissues compared to unreactivated (latent) tissues in untreated (control mice).

### HSV Target Site PCR Amplification

Total genomic DNA (gDNA) was extracted using either the DNeasy Tissue & Blood micro kit for neuronal cultures or the DNeasy Tissue & Blood mini kit for whole TGs. Platinum Pfx DNA polymerase and 5 μl of gDNA were used to PCR amplify the region containing the target site for either HSV1m5 with U_{L}19 primers: forward 5′-CTGGCCGTGGTCGTACATGA (SEQ ID NO: 43) and reverse 5′-TCACCGACATGGGCAACCTT (SEQ ID NO: 44), HSV2m8 with U_{L}30 primers: forward 5′-GAGAACGTGGAGCACGCGTACGGC (SEQ ID NO: 45) and reverse 5′-GGCCCGGTTTGAGACGGTACCAGC (SEQ ID NO: 46), HSV1m4 with ICP0 primers: forward 5′-GACAGCACGGACACGGAACT (SEQ ID NO: 47) and reverse 5′-TCGTCCAGGTCGTCGTCATC (SEQ ID NO: 48), SaCas9/sgRNA_{UL54} (sgRNA13, sgRNA17 and sgRNA26) with U_{L}54 primers: forward 5′-GACCGCATCAGCGAGAGCTT (SEQ ID NO: 49) and reverse 5′-CTCGCAGACACGACTCGAAC (SEQ ID NO: 50), or SaCas9/sgRNA_{UL30} (sgRNA1, and sgRNA10) with U_{L}30-primers: forward 5′-CGGCCATCAAGAAGTACGAG (SEQ ID NO: 51) and reverse 5′-AAGTGGCTCTGGCCTATGTC (SEQ ID NO: 52), with thermocycler conditions of: 94° C. 5 minutes, 40-45 cycles (94° C. 30 seconds, 60° C. 30 seconds, 70° C. 30 seconds), and then 70° C. 5 minutes.

### T7 Endonuclease 1 (T7E1) Assay

The T7 endonuclease assay and quantification to determine the levels of gene disruption were performed as follows. After PCR amplification of target site from HSV genomes, followed by purification using Zymo Research clean and concentrator-5 kit, 300 ng of DNA amplicon was denatured for 10 min at 95° C. and slowly reannealed by cooling down to room temperature. DNA was then digested with 5-10 units of T7 endonuclease for 30-60 min at 37° C. and resolved in an agarose gel. Quantification of gene disruption was performed using ImageJ software and calculated using the formula: 100×(1-[1-fraction cleaved] ½) where fraction cleaved=density of cleaved product/(density of cleaved product+ density of uncleaved product).

ddPCR Quantification

Viral genome quantification by ddPCR was performed using an AAV ITR primer/probe set, and a gB primer/probe set for HSV as described previously. Cell numbers in tissues were quantified by ddPCR using mouse-specific RPP30 primer/probe set: For 5′-GGCGTTCGCAGATTTGGA (SEQ ID NO: 53), Rev 5′-TCCCAGGTGAGCAGCAGTCT (SEQ ID NO: 54), probe 5′-ACCTGAAGGCTCTGCGCGGACTC (SEQ ID NO: 55). In some control ganglia, sporadic samples showed positivity for AAV genomes, although at levels typically >2-3 logs lower than ganglia from treated mice that had received AAV. This can be attributed to low-level contamination of occasional tissue samples.

### Illumina Next Generation Sequencing (NGS)

Next generation sequencing of meganuclease target sites was performed using PCR products generated with the target site-specific primers described above and a MiSeq sequencer.

### Single Cell RNA Analysis

Swiss-Webster mice were latently infected with 10⁵ PFU HSV syn 17+ via the ocular route, and after 60 days injected with one of 4 different AAV serotypes: 1, 8, PHP.S, and Rh10, each carrying a unique fluorescent protein transgene: mScarlet, mEGFP, DsRed.Express2, and TagBFP2 respectively under the CBh promoter. For each serotype three mice were independently injected with 10¹² AAV genomes subcutaneously in the whisker pad (AAV1) or intravenously in the retro-orbital vein (AAV8, PHP.S, and Rh10). Three weeks later, TG and SCG from animals were collected and each tissue (TG or SCG) was pooled from all animals for neuron isolation via enzymatic tissue digest (see above), followed by density gradient centrifugation and enrichment using the Neuron Isolation Kit, which allows untouched neurons to flow through the column while non-neuronal cells remain bound.

Tissue and isolated neurons were maintained in ice cold Neurobasal A medium supplemented with 2% B27 supplement, 1% PenStrep, L-glutamine (500 μM) or PBS throughout the procedure except during the enzymatic tissue digestion steps. Cells were encapsulated and scRNA-seq libraries were prepared in the Genomics Core Facility at the FHCRC using the Chromium Single Cell 3′ Library and Gel Bead Kit v2 from 10× Genomics according to manufacturer instructions. 10× Genomics Single Cell 3′ expression libraries were sequenced on an Illumina HiSeq 2500 running in High-Output mode with a paired end (26 bp×8 bp×98 bp) sequencing strategy. The SCG and TG libraries were pooled and distributed over 8 sequencing lanes. Image analysis and base calling were performed using RTA Version 1.18.66.3. Sequencing reads were processed with the 10× Genomics ‘Cell Ranger’ v2.1.0 and with Seurat v2.3.4. High quality sequence data was obtained from 2,372 purified TG neurons and 2,172 SCG neurons.

### Statistics and Reproducibility

GraphPad Prism 7 software was used for statistical analyses. Comparison of HSV loads was performed with multiple t-test, with alpha=0.05%. Each tissue was analyzed individually, without assuming a consistent SD. HSV and AAV distributions in scRNA-seq experiments were analyzed using the x2 test with alpha=0.05. Because tissue from animals injected with the different AAV serotype/transgene were pooled, cell counts were normalized to input by multiplying cell count by number of animals receiving that serotype divided by total number of animals. The mean and error bars representing the standard deviation are shown on each graph. The findings of the studies were reproduced across the experiments using different experimental set-ups.

RT-ddPCR Quantification of SaCas9, sgRNA, HSV1m5 Expression

AllPrep DNA/RNA kit was used to isolate DNA and RNA from ganglia collected in experiment. SaCas9, sgRNA and HSV2m5 expression was quantified with One-step RT-ddPCR kit using 2 μl of RNA and the following primers/probe set: SaCas9 specific primers: SaCas9 forward 5′-CCGCCCGGAAAGAGATTATT (SEQ ID NO: 56), reverse 5′-CGGAGTTCAGATTGGTCAGTT (SEQ ID NO: 57), and probe [FAM]AGCTGCTGGATCAGATTGCCAAGA [MGB] (SEQ ID NO: 58); Tracr specific primers: TRACR LS forward 5′-TGCCGTGTTTATCTCGTCAACT (SEQ ID NO: 59), reverse 5′-CCCGCCATGCTACTTATCTACTTAA (SEQ ID NO: 60), and probe [FAM]TTGGCGAGATTTTT [MGB] (SEQ ID NO: 61); HSV2m5 specific primers: m5 mega forward 5′-TGGACAGCCTGAGCGAGAA (SEQ ID NO: 62), reverse 5′-GCAGAGACAGAGGAGCAATGTG (SEQ ID NO: 63), and probe [FAM]CGGCCGGTGATTCCTCTGTTTCTAATTC [BHQ] (SEQ ID NO: 64). The cycling steps were as follows: reverse transcription 50° C. 60 minutes, enzyme activation 95° C. 10 minutes, 40 cycles (95° C. 30 seconds, 60° C. 1 minute, 70° C. 30 seconds), and then enzyme deactivation 98° C. 10 minutes.

### Example 4

### HSV-2-Specific Meganucleases Mediate Gene Editing in Mammalian Cells, Are Well-Tolerated in Mice, and Reduce HSV-2 Shedding

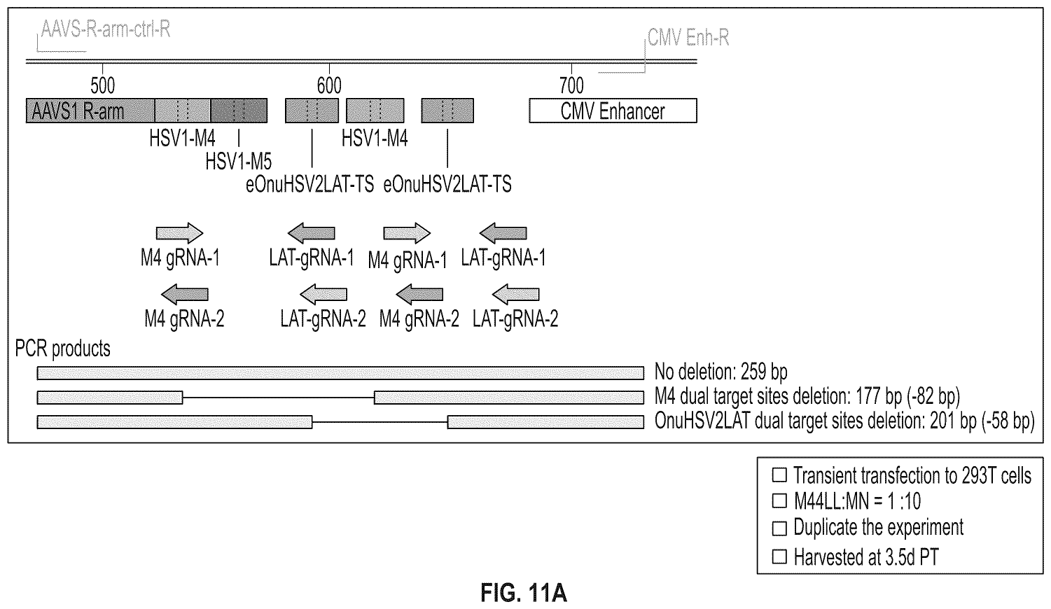

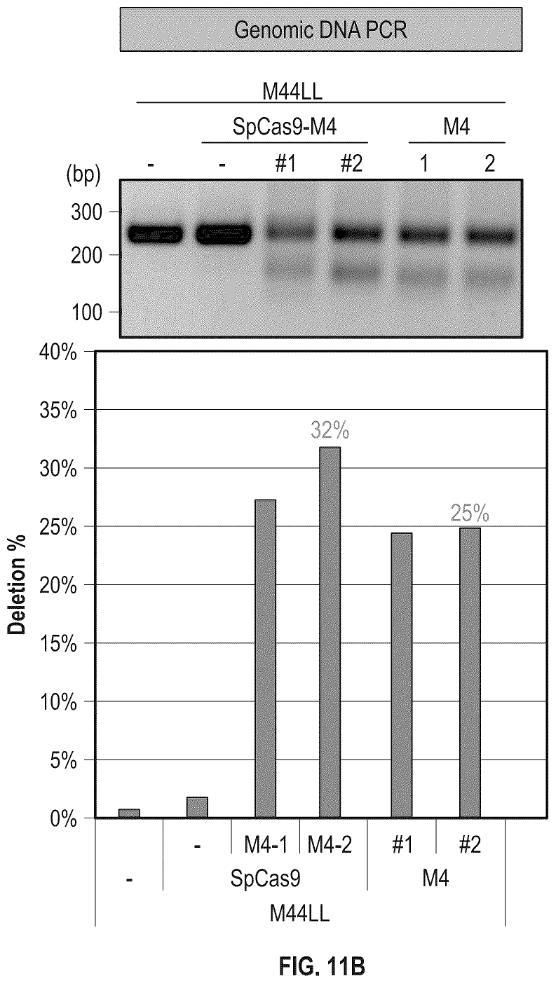

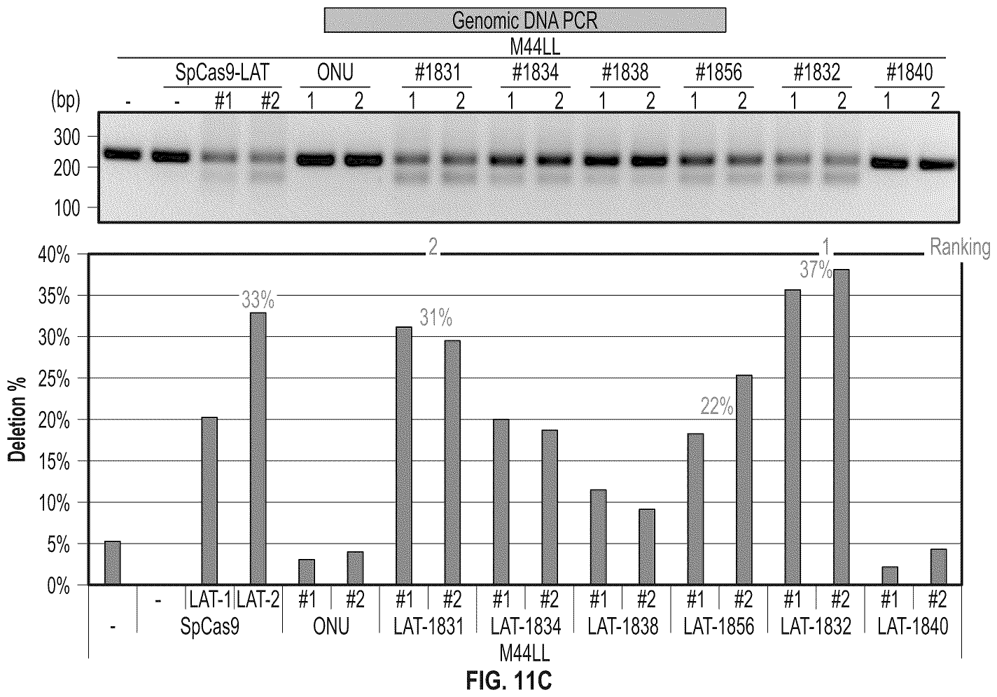

The meganuclease engineering and screening described in Examples 1 and 2 were performed in yeast cells. To confirm that the resulting meganucleases also had activity in mammalian cells, a mammalian reporter system was developed, consisting of a plasmid containing two sites for OnuHSV2a, as well as sites for m4 and CRISPR/Cas9 (LAT-gRNA1 and LATgRNR2) (FIG. 11A). With a functioning enzyme, the sequence between the target sites was removed, and the resulting PCR amplicon was smaller than it was in the uncleaved state. FIGS. 11B and 11C show gels which illustrate enzyme cleavage activity as demonstrated by the smaller PCR amplicon (lower band/shorter bp sequence), in comparison with the uncleaved PCR amplicon (higher band/longer bp sequence). A single higher/longer base pair band indicates no activity (e.g. left 2 lane controls). All experiments were conducted in duplicate. The spCas9 “duplicates” were conducted with two different gRNAs. We then performed dosimetry to quantitate the relative efficiencies of the meganucleases (shown in the bar graphs at the bottom of the panels B and C). All candidates tested worked, as evidenced by the smaller PCR amplicon band (lower band/shorter bp sequence) in the gel. We found that meganucleases 1831 and 1832 had the strongest activity, as evidenced by the highest percent deletion (31% and 37%, respectively). Negative controls (wild type I-Onul and sequence 1840) were negative as expected.

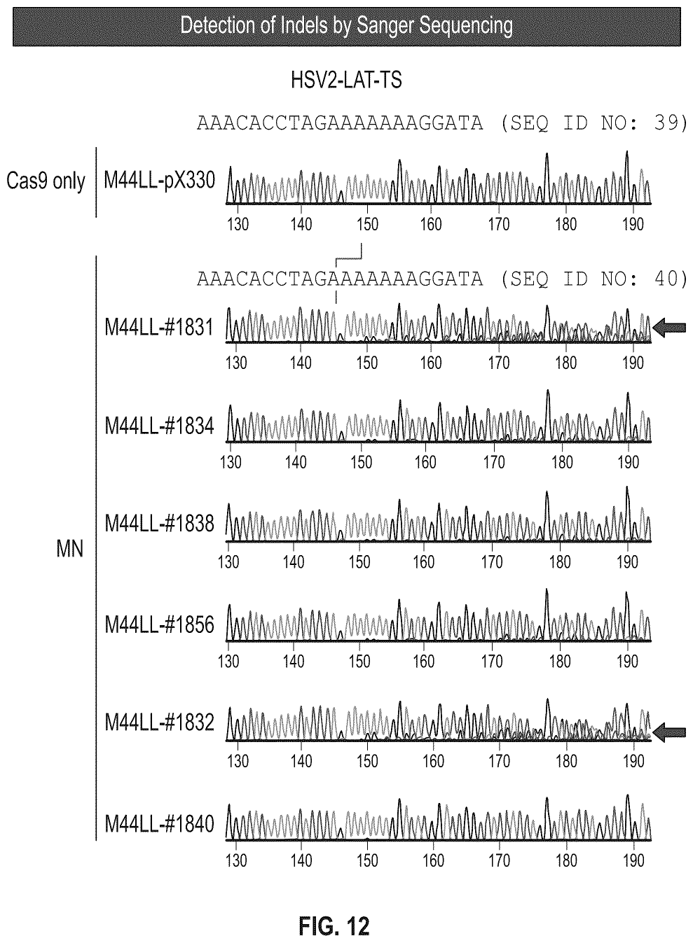

As an alternative approach to confirming the enzyme's activity in mammalian cells, the region of the reporter construct containing the enzyme target sites was PCR amplified, and then the amplicons were analyzed by Sanger sequencing (FIG. 12). Cleavage of the predicted site (shown at the top with the 3-segment line indicating the staggered ends of the cut), is demonstrated by the tracing changing from “clean” with relatively little background (small peaks under the mail tracing), to “dirty” with lots of subdominant peaks as indicated by the arrows. Consistent with our results from the gel analysis of the previous section, cleavage was most efficient with meganucleases 1831 and 1832, wherein numerous subdominant peaks appeared, demonstrating significant by these variants.

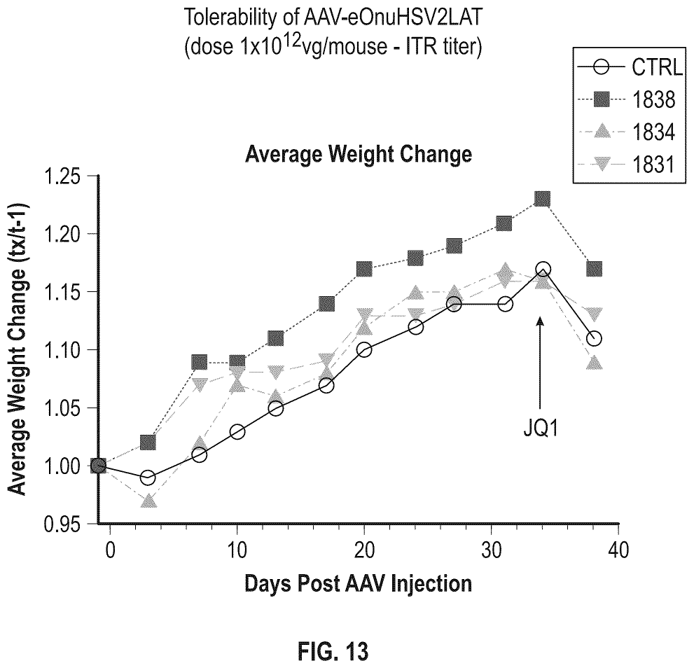

To confirm that I-Onul-derived meganucleases targeting HSV-2 would be tolerated in vivo, meganucleases 1831, 1834, or 1838 were administered to mice, using 1×10¹² vector genomes (vg) AAV9 with the meganuclease under control of the CbH promoter, and followed the weights of the animals (FIG. 13). Administration of m4 under these conditions results in slowed or absent weight gain after therapy. Unlike animals that receive m4 under the same conditions (not shown), animals receiving the Onu-based enzymes gain weight normally after therapy, suggesting the Onu-based enzymes are better tolerated with no overt signs of toxicity.

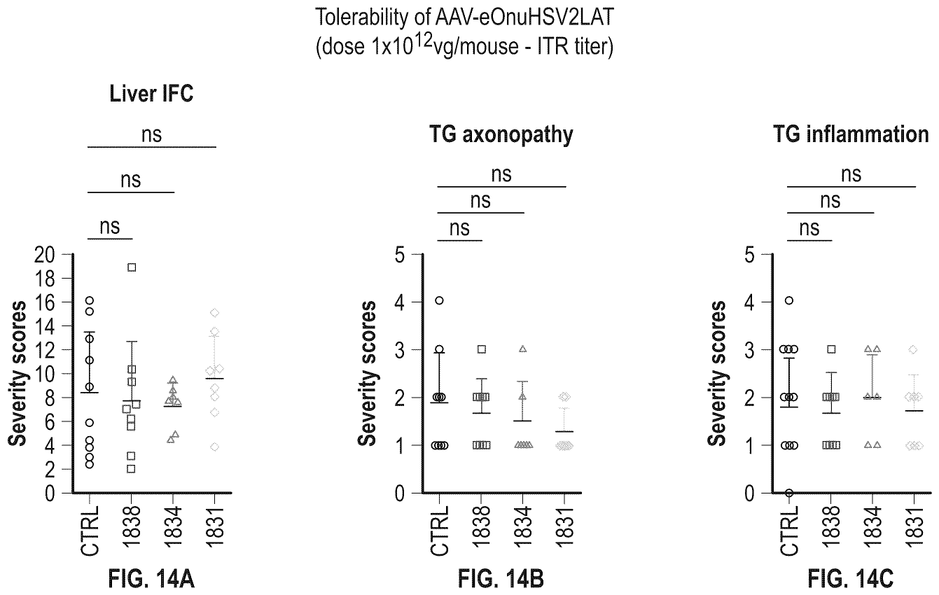

As an additional approach to confirm that Onu-derived meganucleases targeting HSV-2 would be tolerated in vivo, meganucleases 1831, 1834, or 1838 were administered to mice, using 1×10¹² vector genomes (vg) AAV9 with the meganuclease under control of the CbH promoter, and evaluated liver inflammatory cell foci (IFC), TG axonopathy, and TG inflammation (FIG. 14). Administration of m4 under these conditions results in an increase in ICF, TG axonopathy score, and TG inflammation score. Unlike animals which received m4 under the same conditions (not shown), no detectable liver toxicity or neurotoxicity was observed with meganucleases 1831, 1834, or 1838.

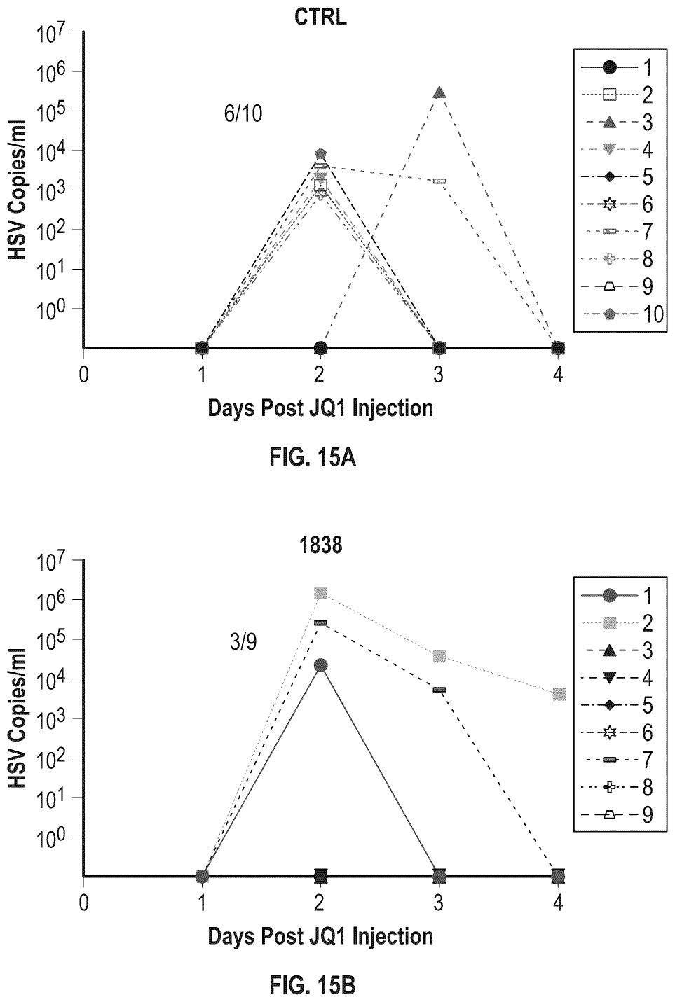

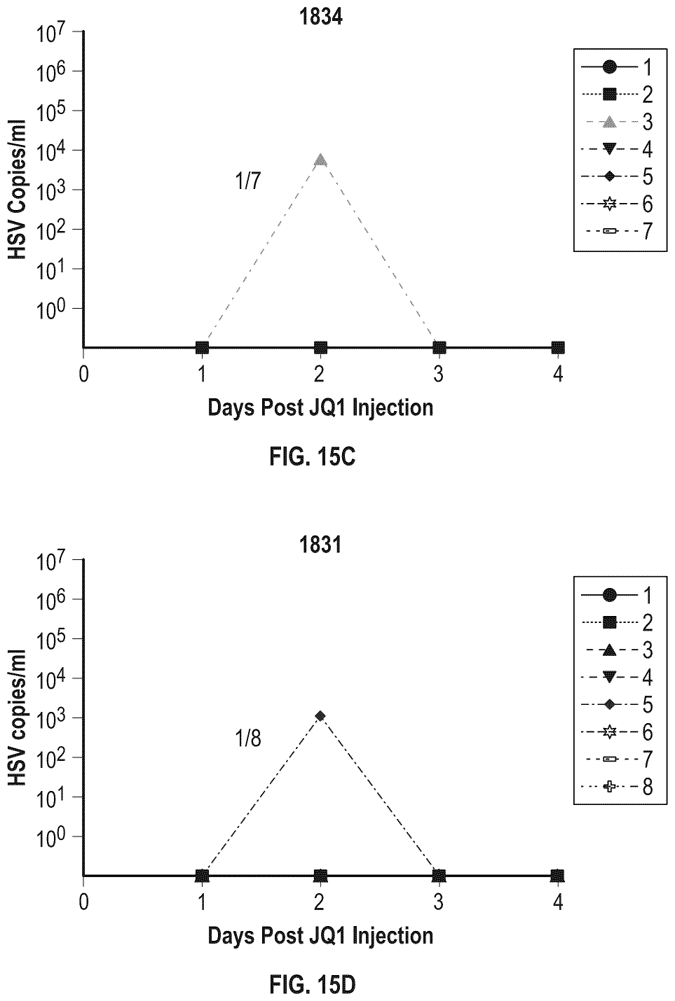

To determine whether treatment with I-Onul-derived meganucelases can successfully reduce HSV-2 shedding from latently infected animals, mice with latent HSV-2 infection were treated with 1×10¹² vector genomes (vg) AAV9 with meganuclease 1831, 1834, or 1838 under control of the CbH promoter, or left untreated (controls, CTRL, FIG. 15). One month later, HSV-2 shedding was induced using the bromodomain inhibitor JQ1. In the control untreated group, 6 of the 10 control animals shed HSV at the indicated levels after JQ1. In contrast, only 3/9 animals shed when treated with 1838; only a single animal shed after treatment with 1834; only a single animal shed after treatment with 1831. These results generally recapitulate the relative efficiencies of the enzymes in the reporter experiments (FIG. 11 and FIG. 12, 1831>1834>1838), and confirm that the I-OnuI-derived meganucleases have anti-HSV-2 activity in vivo.

Embodiment 1. A composition comprising a plurality of one or more viral vectors, wherein each of the one or more viral vectors includes a sequence encoding an HSV-2-specific meganuclease.

Embodiment 2. The composition of Embodiment 1, wherein the one or more viral vectors is a self-complementary adeno-associated virus (scAAV), a single-stranded adeno-associated virus (ssAAV), or a combination thereof.

Embodiment 3. The composition of Embodiment 2, wherein the ssAAV is ssAAV9, ssAAV-Dj/8, ssAAV-rh10, ssAAV8, ssAAV1, another serotype adeno-associated virus, or a combination thereof.

Embodiment 4. The composition of Embodiment 2, wherein the scAAV is scAAV9, scAAV-Dj/8, scAAV-rh10, scAAV8, scAAV1, another serotype adeno-associated virus, or a combination thereof.

Embodiment 5. The composition of any one of Embodiments 1-4, wherein the sequence encoding the HSV-2-specific meganuclease is configured to encode an HSV-2-specific meganuclease which targets one or more HSV-2 genes essential for replication.

Embodiment 6. The composition of any one of Embodiments 1-5, wherein the sequence encoding the HSV-2-specific meganuclease is configured to encode an HSV-2-specific meganuclease which induces one or more DNA double strand breaks.

Embodiment 7. The composition of any one of Embodiments 1-6, wherein the HSV-2-specific meganuclease is configured to induce one or more DNA double strand breaks.

Embodiment 8. The composition of any one of Embodiments 1-7, wherein the HSV-2-specific meganuclease is configured to induce two DNA double strand breaks.

Embodiment 9. The composition of any one of Embodiments 1-8, wherein the HSV-2-specific meganuclease is configured to target one or more HSV-2 genes essential for replication.

Embodiment 10. The composition of any one of Embodiments 1-9, wherein the HSV-2-specific meganuclease comprises a sequence as set forth in SEQ ID NOs: 1-6.

Embodiment 11. The composition of any one of Embodiments 1-10, wherein the HSV-2-specific meganuclease comprises SEQ ID NO: 1 or 2.

Embodiment 12. The composition of any one of Embodiments 1-9, wherein the HSV-2-specific meganuclease comprises a sequence as set forth in SEQ ID NOs: 7-12.

Embodiment 13. The composition of any one of Embodiments 1-12, wherein the HSV-2-specific meganuclease is configured to target one or more sequences as set forth in SEQ ID NOs: 13 and 14.

Embodiment 14. The composition of any one of Embodiments 1-13, wherein the one or more viral vectors further includes a regulatory sequence.

Embodiment 15. The composition of Embodiment 14, wherein the regulatory sequence comprises a sequence as set forth in SEQ ID NO: 42.

Embodiment 16. The composition of any one of Embodiments 1-15, wherein the plurality of one or more viral vectors comprises one to three scAAVs, wherein each scAAV is a different serotype, and wherein each scAAV includes a sequence, which is the same or different, encoding an HSV-2-specific meganuclease.

Embodiment 17. A pharmaceutical composition, comprising the composition of any one of Embodiments 1-16, and a pharmaceutically acceptable carrier or excipient.

Embodiment 18. A method of reducing or eliminating latent HSV-2 or HSV-2 reactivation in a cell, comprising administering the composition of any one of Embodiments 1-16 to the cell.

Embodiment 19. The method of Embodiment 18, comprising administering the composition to a cell in an amount effective to reduce or eliminate latent HSV-2 or HSV-2 reactivation in the cell.

Embodiment 20. The method of Embodiment 18 or 19, wherein the cell is a mammalian cell.

Embodiment 21. The method of any one of Embodiments 18-20, wherein the cell is a neuron.

Embodiment 22. The method of any one of Embodiments 18-21, wherein the cell is a sensory ganglia cell, an autonomic ganglia cell, or a combination thereof.

Embodiment 23. The method of any one of Embodiments 18-22, wherein the cell is a superior cervical ganglia cell, a trigeminal ganglia cell, a dorsal root ganglia cell, a major pelvic ganglia cell, another HSV-2 infected cell, or a combination thereof.

Embodiment 24. A method of reducing or eliminating latent HSV-2 or HSV-2 reactivation in a subject, comprising administering to the subject the composition of any one of Embodiments 1-16 in an amount effective to reduce or eliminate latent HSV-2 or HSV-2 reactivation in the subject.

Embodiment 25. A method of reducing or eliminating latent HSV-2 or HSV-2 reactivation in a subject, comprising administering to the subject the pharmaceutical composition of Embodiment 17.

Embodiment 26. The method of Embodiment 24 or 25, wherein the administering is by a subcutaneous injection or intramuscular injection.

Embodiment 27. The method of any one of Embodiments 24-26, wherein the subject is a mammal.

Embodiment 28. The method of any one of Embodiments 24-27, wherein the subject is a human.

While illustrative embodiments have been illustrated and described, it will be appreciated that various changes can be made therein without departing from the spirit and scope of the invention.

1. A composition comprising a plurality of one or more viral vectors, wherein each of the one or more viral vectors includes a sequence encoding an HSV-2-specific meganuclease.

2. The composition of claim 1, wherein the one or more viral vectors is a self-complementary adeno-associated virus (scAAV), a single-stranded adeno-associated virus (ssAAV), or a combination thereof.

3. The composition of claim 2, wherein the ssAAV is ssAAV9, ssAAV-Dj/8, ssAAV-rh10, ssAAV8, ssAAV1, another serotype adeno-associated virus, or a combination thereof.

4. The composition of claim 2, wherein the scAAV is scAAV9, scAAV-Dj/8, scAAV-rh10, scAAV8, scAAV1, another serotype adeno-associated virus, or a combination thereof.

5. The composition of claim 1, wherein the sequence encoding the HSV-2-specific meganuclease is configured to encode an HSV-2-specific meganuclease which targets one or more HSV-2 genes essential for replication.

6. The composition of claim 1, wherein the sequence encoding the HSV-2-specific meganuclease is configured to encode an HSV-2-specific meganuclease which induces one or more DNA double strand breaks.

7. The composition of claim 1, wherein the HSV-2-specific meganuclease is configured to induce one or more DNA double strand breaks.

8. The composition of claim 1, wherein the HSV-2-specific meganuclease is configured to induce two DNA double strand breaks.

9. The composition of claim 1, wherein the HSV-2-specific meganuclease is configured to target one or more HSV-2 genes essential for replication.

10. The composition of claim 1, wherein the HSV-2-specific meganuclease comprises a sequence as set forth in SEQ ID NOs: 1-6.

11. The composition of claim 1, wherein the HSV-2-specific meganuclease comprises SEQ ID NO: 1 or 2.

12. The composition of claim 1, wherein the HSV-2-specific meganuclease comprises a sequence as set forth in SEQ ID NOs: 7-12.

13. The composition of claim 1, wherein the HSV-2-specific meganuclease is configured to target one or more sequences as set forth in SEQ ID NOs: 13 and 14.

14. The composition of claim 1, wherein the one or more viral vectors further includes a regulatory sequence.

15. The composition of claim 14, wherein the regulatory sequence comprises a sequence as set forth in SEQ ID NO: 42.

16. The composition of claim 1, wherein the plurality of one or more viral vectors comprises one to three scAAVs, wherein each scAAV is a different serotype, and wherein each scAAV includes a sequence, which is the same or different, encoding an HSV-2-specific meganuclease.

17. A pharmaceutical composition, comprising the composition of claim 1, and a pharmaceutically acceptable carrier or excipient.

18. A method of reducing or eliminating latent HSV-2 or HSV-2 reactivation in a cell, comprising administering the composition of claim 1 to the cell.

19. The method of claim 18, comprising administering the composition to a cell in an amount effective to reduce or eliminate latent HSV-2 or HSV-2 reactivation in the cell.

20. The method of claim 18, wherein the cell is a mammalian cell.

21. The method of claim 18, wherein the cell is a neuron.

22. The method of claim 18, wherein the cell is a sensory ganglia cell, an autonomic ganglia cell, or a combination thereof.

23. The method of claim 18, wherein the cell is a superior cervical ganglia cell, a trigeminal ganglia cell, a dorsal root ganglia cell, a major pelvic ganglia cell, another HSV-2 infected cell, or a combination thereof.

24. A method of reducing or eliminating latent HSV-2 or HSV-2 reactivation in a subject, comprising administering to the subject the composition of claim 1 in an amount effective to reduce or eliminate latent HSV-2 or HSV-2 reactivation in the subject.

25. A method of reducing or eliminating latent HSV-2 or HSV-2 reactivation in a subject, comprising administering to the subject the pharmaceutical composition of claim 17.

26. The method of claim 24, wherein the administering is by a subcutaneous injection or intramuscular injection.

27. The method of claim 24, wherein the subject is a mammal.

28. The method of claim 24, wherein the subject is a human.
